(19) United States
(12) Patent Application Publication
(10) Pub. No.: US 2021/0161999 A1
(43) Pub. Date: Jun. 3, 2021

(54) COMPOSITIONS AND METHODS RELATED TO OVERCOMING INNATE IMMUNE BARRIERS TO CANCER IMMUNOTHERAPY

(71) Applicant: Health Research, Inc., Buffalo, NY (US)

(72) Inventors: Brahm SEGAL, Williamsville, NY (US); Kelly SINGEL, Bethesda, MD (US)

(21) Appl. No.: 17/266,993

(22) PCT Filed: Aug. 9, 2019
(86) PCT No.: PCT/US19/45880

§ 371 Date: Feb. 8, 2021

Related U.S. Application Data
(60) Provisional application No. 62/716,496, filed on Aug. 9, 2018.

(51) Int. Cl. A61K 38/12; A61K 39/395; A61P 35/00; G01N 33/50

(52) U.S. Cl. CPC A61K 38/12; A61K 39/3955; A61K 2039/505; G01N 33/505; G01N 33/5011; A61P 35/00

(57) ABSTRACT

Provided are methods for identifying and treating individuals who have cancer, and also have immunosuppressive neutrophils. The method of treating includes administering one or more drugs that inhibit formation of immunosuppressive neutrophils. Cancer patients can be identified, and selected for treatment, based on a positive result obtained by exposing a biological sample from the patient to normal neutrophils, and subsequently exposing the neutrophils to T cells, and measuring activation of T the cells. Reduced activation of the T cells relative to a control provides an indication that the individual has the immunosuppressive neutrophils, and is a candidate to receive the drug. The drug administered to the cancer patient functions to inhibit SNARE-dependent exocytosis, or inhibits NADPH oxidase, or inhibits complement signaling. The method further includes administering to the individual an immune checkpoint inhibitor, which may increase the efficacy of the checkpoint inhibitor.

### CROSS-REFERENCE TO RELATED APPLICATIONS

This application claims priority to U.S. provisional patent application no. 62/716,496, filed Aug. 9, 2018, the disclosure of which in incorporated herein by reference.

### STATEMENT REGARDING FEDERAL SPONSORED RESEARCH OR DEVELOPMENT

This invention was made with government support under grant numbers R01CA188900, T32CA085183, and P30CA016056 awarded by the National Institutes of Health. The government has certain rights in the invention.

### FIELD

The present disclosure relates generally to modulating immune responses and more specifically to inhibiting immunosuppressive neutrophils.

### BACKGROUND

Immunotherapy has revolutionized cancer therapy. However, multiple barriers exist that abrogate anti-tumor immunity. There is thus an ongoing and unmet need to provide compositions and methods related to improving cancer patient outcomes. The present disclosure is pertinent to these needs.

### BRIEF SUMMARY

The present disclosure provides methods for identifying and treating individuals who have cancer, and also have immunosuppressive neutrophils. In embodiments, the disclosure provides a method of providing a treatment for a cancer patient with one or more drugs that inhibit formation of immunosuppressive neutrophils. In one embodiment, this method comprises selecting a cancer patient based on a positive result obtained by exposing a biological sample from the patient to normal neutrophils, and subsequently exposing the neutrophils to T cells, and measuring activation of the T cells. Reduced activation of the T cells relative to a control comprises an indication that the individual has the immunosuppressive neutrophils, and is a candidate to receive the drug. In embodiments, the method further includes administering the drug to the individual. In embodiments, the biological sample comprises a pleural effusion. In embodiments, an ascites supernatant can be used. In non-limiting embodiments, the drug administered to the cancer patient functions to inhibit SNARE-dependent exocytosis, or inhibits NADPH oxidase, or inhibits complement signaling. In certain embodiments, the drug administered to the individual comprises a SNARE domain of syntaxin-4 or an N-terminal domain of SNAP23, or comprises a peptide or modified peptide that selectively binds to native C3, and/or to C3 bioactive fragments selected from C3b, iC3b and C3c. In embodiments, the peptide or peptide derivative comprises compstatin, or a compstatin derivative, such as Cp40, PEGylated Cp40, or AMY-101. In embodiments, methods of the disclosure further comprise administering to the individual an immune checkpoint inhibitor. In embodiments, the drug that inhibits formation of immunosuppressive neutrophils increases the efficacy of the immune checkpoint inhibitor. In embodiments, the combination of the drug that inhibits formation of immunosuppressive neutrophils and the immune checkpoint inhibitor is more effective in treating the cancer than administering either the drug or the immune checkpoint inhibitor alone.

In embodiments, the individual identified and/or treated as described herein has ovarian cancer, but the methods of this disclosure are expected to be suitable to identify any cancer patient that has immunosuppressive neutrophils, and providing a treatment based at least in part on this identification. This approach is supported by the discovery of a neutrophil suppressor phenotype when neutrophils and stimulated T cells were cocultured with pleural fluid from patients with a number of metastatic cancers (e.g., lung, breast, pancreatic), which was abrogated by Cp40 as a representative compound.

Results presented in this disclosure demonstrate, among other findings, patients with newly diagnosed advanced Epithelial ovarian cancer (EOC), circulating neutrophils (PMN) are not intrinsically suppressive, but acquire a suppressor phenotype once recruited to the tumor microenvironment (TME). Ascites supernatants induced PMN to suppress stimulated T cell proliferation, activation, and cytokine responses, but did not affect cytotoxic T lymphocytes (CTL) activity. These results show that while the PMN suppressor phenotype will not affect the CTL activity of effector T cells, the phenotype will prevent the expansion of these CTL in the TME. The PMN suppressor phenotype inhibited T cell proliferation in stimulated naïve, central memory, and effector memory T cells, as well as in CTL with engineered T-cell receptors (TCRs). Mature PMN fully recapitulated the suppressor phenotype attributed to granulocytic myeloid-derived suppressor cells (MDSC) and N2 tumor-associated PMN (TAN). Although the distinction between granulocytic MDSC and N2 TAN is debated, the common feature is a circulating population of suppressor granulocytes, while the PMN suppressor phenotype is identified in this disclosure is acquired in the TME and dependent on several PMN effector functions. In addition, targeting TGF-beta signaling did not abrogate the phenotype, which indicates that this newly identified PMN suppressor phenotype is distinct from TGF-beta-driven N2 polarization. Moreover, malignant effusions from patients with various metastatic cancers also induced the C3-dependent PMN suppressor phenotype, supporting the generalizability of these results. Together, these results point to mature PMN impairing T cell expansion and activation in the TME and provide a number of therapeutic targets to abrogate this barrier to anti-tumor immunity.

It will be recognized that the present disclosure has the benefit of using ascites from patients, rather than tumor-conditioned media or tumor-bearing mice. The results of this disclosure indicate soluble, heat-labile protein(s), specifically complement, in ascites inducing the PMN suppressor phenotype. We observed negligible to no effect of PMN alone in T cell suppression studies, while PMN combined with ascites supernatants resulted in dramatic suppression of T cell proliferation, frequently to unstimulated levels. The effect was observed with both anti-CD3/CD28 microbeads and soluble anti-CD3/CD28 antibodies (Ab). Finally, we used a high standard for defining suppression as ≤1 log₁₀ reduction in anti-CD3/CD28-stimulated T cell proliferation that was well above any background effects observed with PMN alone.

PMN can have considerable heterogeneity and plasticity, with the potential to enhance or suppress anti-tumor immunity (43, 57). Coffelt et al. (58) showed that PMN suppressed CTL responses, and depletion of IL-17 or G-CSF abrogated the T cell suppressive phenotype in a mammary tumor model. PMN have also enhanced mammary tumor metastasis to lungs (59). Targeting CXCR2, which mediates PMN recruitment, suppressed tumorigenesis and metastasis in mice (60), and dual targeting of CXCR2 and CCR2 enhanced responses to chemotherapy (61). Activated PMN can also kill tumor cells (62). Eruslanov et al. (63) showed that TAN from early stage lung cancer enhanced T cell responses. By contrast, our results show that PMN acquired a suppressor phenotype once exposed to the TME. Thus, and without intending to be constrained by any particular theory, it is considered that the programming of PMN to a pro- or anti-tumorigenic phenotype depends on cues within the TME that include tumor-derived factors and products of inflammation and injury.

We also observed that post-operative drainage fluid induced the PMN suppressor phenotype similar to paired pre-operative ascites. This finding was observed in patients with RO and optimal debulking surgeries. Since primary surgery for advanced EOC is non-curative, post-surgical immunosuppression is expected to be clinically relevant. The concept of the pro-tumorigenic effect of surgery is indirectly supported by short delays in adjuvant chemotherapy after primary surgery for EOC that correlate with shorter PFS and OS. These findings indicate that the expansion of suppressive PMN, both immature and mature, can occur in response to multiple insults and through distinct signaling pathways. The induction of suppressive PMN is thus also a strategy to limit tissue injury or avert autoimmunity by restraining T cell responses; while in the TME, these same pathways are indicated to impede anti-tumor immunity, a finding that provides at least in part a basis for the present disclosure.

### BRIEF DESCRIPTION OF THE FIGURES

FIG. 1. Ovarian cancer ascites induces circulating patient PMN to become suppressive. A) The proportion of circulating WBC populations in a healthy donor (n=1), control female patients undergoing surgery for a benign peritoneal mass (n=3), and patients undergoing surgery for newly diagnosed HGSOC (n=3) are similar, but differ significantly from WBC populations in paired HGSOC ascites (n=3). B-D) Cytologic analysis of Wright Giemsa-stained cytospins of ascites from newly diagnosed HGSOC (n=10). B) Representative image showing mature PMN (N), monocytes/macrophages (M), lymphocytes (L), and tumor cells (C). All PMN were morphologically mature with characteristic segmented nuclei. C) WBC proportions were quantified: PMN 4-52%, monocytes/macrophage 17-87%, and lymphocytes 8-69%. D) Mean PMN-to-lymphocyte ratio was 1.03 [95% CI 0.21-1.8, SEM 0.4]. E-F) T cells (CD3⁺) and PMN were isolated from patient blood and used in autologous coculture at 1:1 based on data in (D) (n=4). PMN and/or ascites supernatants (ASC; 50% final well volume) were added to anti-CD3/CD28-stimulated T cells. After 72 h of coculture, T cell proliferation was measured by [³H] thymidine incorporation (16-18 h). E) HGSOC patient circulating PMN were negligibly T cell suppressive. F) ASC are not suppressive alone but induce patient PMN to suppress stimulated T cell proliferation by a factor of 2.08 log₁₀ [95% CI 1.26-2.90]. Symbols represent individual samples (n) and bars represent SEM. Statistical comparisons were by ANOVA with Tukey post-test, (*, p<0.05; **, p<0.01; ***, p<0.001; ns, not significant).

FIG. 2. Suppressed T cells are viable and responsive to secondary stimulation. T cells (CD3⁺) and PMN were used in autologous coculture at 1:1. PMN and/or ascites supernatants (ASC; 50% final well volume) were added to anti-CD3/CD28-stimulated T cells. After 72 h of coculture, T cell proliferation was measured by [³H] thymidine incorporation (16-18 h). A) Results are consistent with soluble anti-CD3/CD28 Ab or anti-CD3/CD28 microbeads as T cell stimulus. B) ASC (n=31) were stratified into three categories based on the induction of a PMN suppressor phenotype, where x equals a reduction in proliferation as compared to anti-CD3/CD28-stimulated T cells alone: suppressors (SUPP, line 3; x≥1 log₁₀), intermediate suppressors (INTERMED, line 2; 0.5 log₁₀≤x<1 log₁₀), and non-suppressors (NON-SUPP, line 1; x<0.5 log₁₀). SUPP-A and B illustrate that a subset of ascites supernatants induced PMN suppressors x≥2 log₁₀. Bars are representative. C-E) PMN suppressor phenotype fully suppressed anti-CD3/CD28-stimulated C) naïve (CD3⁺CD45RA⁺RO^{neg}CD62L⁺), D) central memory (CD3⁺CD45RA^{neg}RO³⁰ CD62L⁺), and E) effector memory (CD3⁺CD45RA^{neg}RO⁺CD62L^{neg}) T cell populations (n=2). F) T cells were annexin-V negative (>70%) after 72 h coculture with ASC (n=3) and/or PMN. Fas ligand was added to stimulated T cells as a positive control for apoptosis. G) Stimulated T cell proliferation was suppressed after 72 h with ASC and PMN, but H) restored after ASC removal and anti-CD3/CD28-restimulation (n=5). I) Addition of rIL-2 (100 IU) at 48 h did not rescue T cell proliferation, as assessed at 72 h. Symbols represent individual samples (n) and bars represent SEM. Statistical comparisons were by ANOVA with Tukey post-test (*, p<0.05; **, p<0.01; ***, p<0.001; ns, not significant). Results were consistent between CD4⁺ and CD8⁺ T cells.

FIG. 3. PMN suppressor phenotype requires contact between PMN and T cells, complement C3 activation, and complement receptor 3. T cells (CD3⁺) and PMN were used in autologous coculture at 1:1. PMN and/or ascites supernatants (ASC; 50% final well volume) were added to anti-CD3/CD28-stimulated T cells. After 72 h of coculture, T cell proliferation was measured by [³H] thymidine incorporation (16-18 h). A) T cells were stimulated with anti-CD3/CD28 in the bottom chamber. PMN and ASC added to the transwell insert did not suppress T cell proliferation, suggesting that suppression is contact-dependent (n=4). B) T cells treated with anti-ICAM-1 Ab (1-10 μg) for lh prior to coculture had no effect on proliferation. PMN treated with anti-CD11b Ab for 1 h prior to coculture abrogated the suppressor phenotype. Treatment of T cells or PMN with IgG1 isotype (1-10 μg) had no effect on proliferation (n=5). C) PMN pretreated with C3b or iC3b (40-160 μg/mL) prior to coculture were unable to induce the PMN suppressor phenotype. D) ASC were heat-inactivated (HI-ASC; 56° C., 1 h) prior to coculture and abrogated the PMN suppressor phenotype (n=5). E-H) Two formulations of compstatin, CS and Cp40, were used to inhibit C3 activation. E-G) Addition of CS (250 μM) to ASC (CS-ASC) 2 h prior to coculture with PMN and T cells abrogated the PMN suppressor phenotype (n=27). H) Addition of Cp40 (20 μM) to ASC (Cp40-ASC) 2 h prior to coculture also abrogated the PMN suppressor phenotype, while scramble peptide (SCR-ASC, 20 μM) had no effect (n=10). I) A titration study showed that 5 μM Cp40 was sufficient to fully abrogate the PMN suppressor phenotype (n=3). J) ASC were pretreated with neutralizing Ab anti-05 or C7, or with OmCI, a peptide inhibitor of C5, prior to coculture. Anti-05 and OmCI partially abrogated the PMN suppressor phenotype, as compared to their respective controls, whereas anti-C7 did not affect the suppressor phenotype. K) Malignant effusions (ME), including pelural fluid and ascites from patients with a number of metastatic cancers induced the PMN suppressor phenotype, which was abrogated by Cp40-treatment in all of the tested samples (n=7; see Table 3). Symbols represent individual samples (n) and bars represent SEM. Statistical comparisons were by ANOVA with Tukey post-test or by Mann-Whitney (*, p<0.05; **, p<0.01; ***, p<0.001; ns, not significant).

FIG. 4. PMN suppressor phenotype requires SNARE transport and Ca²⁺ mobilization, and is abrogated by desensitization with fMLF. A-F) PMN were treated with media, fMLF (100 nM), ASC (n=3), or heat-inactivated ASC (HI-ASC; n=3) and assessed for markers of membrane fusion with primary (CD63), secondary and tertiary (CD66b) granules, and secretory vesicles (CD35) at 0, 30, and 60 min. PMN were gated on CD45⁺CD15⁺. A, C, E) The MFI overlays are representative; unstimulated PMN in media, grey solid; fMLF, black dashed line; ASC, green line; HI-ASC, purple line. B, D, F) MFI quantification. G-I) T cells (CD3⁺) and PMN were used in autologous coculture at 1:1. PMN and/or ascites supernatants (ASC; 50% final well volume) were added to anti-CD3/CD28-stimulated T cells. After 72 h of coculture, T cell proliferation was measured by [³H] thymidine incorporation (16-18 h). G) Pretreatment with brefeldin-A (BFA; 1-10 μg/mL) or ER export inhibitor 1 (Exol; 20-75 μM) abrogated the suppressor phenotype, indicating a requirement for exocytosis (n=3). H) PMN pretreated with TAT-SNAP23 (0.6 μg) or TAT-SYN4 (0.6 μg) abrogated the PMN suppressor phenotype. TAT-GST (0.6 μg) used as a specificity control had no effect (n=3). I) PMN pretreated with fMLF (100 nM), thapsigargin (THG, 1 μM), or diphenyleneiodonium (DPI, 1 μM) abrogated the PMN suppressor phenotype (n=7). Symbols represent individual samples (n) and bars represent SEM. Statistical comparisons were by ANOVA with Tukey post-test (*, p<0.05; **, p<0.01; ***, p<0.001; ns, not significant).

FIG. 5. Ascites induces robust de novo protein synthesis in PMN, which is required for the suppressor phenotype. A) PMN pretreated with puromycin (1 μg) for lh prior to coculture abrogated the PMN suppressor phenotype, while D-actinomycin (1 μg) pretreatment had more variable effects (n=8). B-D) PMN were exposed to media, ASC, or proteinase K-digested ASC (PK-ASC) for 30 or 60 min in 5 replicates per condition per time point. PMN were washed and frozen as dry pellets for proteomics analysis. B) Heat-map showing that the protein profiles of PMN exposed to ASC have higher Z-scores than either PMN exposed to media or PK-ASC at 30 and 60 min. C) The number of changed proteins (y-axis) in PMN exposed to ASC is significantly greater than in PMN exposed to PK-ASC (p=0.02); there was no significant difference between 30 or 60 min. D) Gene ontology analysis shows that ASC induced new synthesis of multiple classes of proteins in PMN. Symbols represent individual samples (n) and bars represent SEM. Statistical comparisons were by ANOVA with Tukey post-test (*, p<0.05; **, p<0.01; ***, p<0.001; ns, not significant).

FIG. 6. Combination of ascites and PMN prevents T cell activation and is independent of exhaustion. T cells (CD3⁺) and PMN were used in autologous coculture at 1:1. PMN and/or ascites supernatants (ASC; 50% final well volume) were added to anti-CD3/CD28-stimulated T cells. At 24, 48, and 72 h, T cells were analyzed for surface and intracellular expression of markers for activation, co-stimulation, and function. Surface expression of A) CD62L, B) CD69, C) CD4OL, and D) CD107a were evaluated at baseline (on y-axis) and at 24, 48, and 72 h (n=2). E-M) PD-1, LAG-3, and CTLA-4 expression was evaluated (n=4). CD8⁺ T cells at 72 h are represented here in representative MFI overlays (E-G) and quantification (H-J); unstimulated T cells in media, grey solid; anti-CD3/CD28-stimulated, black dashed line; anti-CD3/CD28-stimulated+PMN, purple solid line; anti-CD3/CD28-stimulated+ASC, orange solid line; anti-CD3/CD28-stimulated+ASC+PMN, green solid line. K-M) Stimulated T cell expression of PD-1 and LAG-3 after coculture with Cp40-ASC and PMN was unaffected as compared to unstimulated, but CTLA-4 showed an upwards trend (n=6). N) At 72 h, intracellular expression of IFN-gamma was reduced as compared to stimulated alone (n=3). O-P) Combination of ASC and PMN reduced anti-CD3/CD28-stimulated T cell IL-2 levels (pg/mL) in supernatants to ND after 24 (O) and 72 h (P) of coculture (n=4). Background levels of PMN or ASC were ND; ND=non-detectable. Q) CTL activity of NY-ESO-1₁₅₇₋₁₆₅-specific CD8⁺ T cells directed at SK29 target cells pulsed with the NY-ESO-1 peptide was unaffected by coculture with ASC and/or PMN (n=3). Symbols represent individual samples (n) and bars represent SEM. Statistical comparisons were by Mann-Whitney (*, p<0.05; **, p<0.01; ***, p<0.001; ns, not significant). Results were consistent between CD4⁺ and CD8⁺ T cells.

FIG. 7. PMN suppressor phenotype inhibits engineered effector T cell activation but not antigen-specific cytotoxicity. Engineered CTL expressing NYESO1-specific TCR (TCR-CTL) are candidates for adoptive cell therapy in EOC. TCR-CTL and PMN were used in coculture at 1:1. PMN and/or ascites supernatants (ASC; 50% final well volume) were added to anti-CD3/CD28-stimulated TCR-CTL. After 72 h of coculture (A) or after rIL-2 (100 IU) addition at 48 h (B), CTL proliferation was measured by [³H] thymidine incorporation (16-18 h) (n=3). A) ASC and MD-ASC rendered PMN suppressive to TCR-CTL, while PK-ASC had no effect. B) Addition of rIL-2 reversed suppression. C) IFN-gamma expression was reduced with PMN and/or ASC or PK-ASC, as measured by flow cytometry after 72 h of coculture (n=2). Symbols represent individual samples (n) and bars represent SEM. Statistical comparisons were by ANOVA with Tukey post-test (*, p<0.05; **, p<0.01; ***, p<0.001; ns, not significant).

FIG. 8. Mature PMN in control blood and paired blood and ascites from patients with advanced HGSOC are CD33^{mid} (CD11b⁺CD33^{mid}CD15⁺CD14⁻DR⁻) and monocytes/macrophages are CD33^{high} (CD11b⁺CD33^{hi}CD15⁻CD14⁺DR⁺). A) Gating strategy. Representative raw data from B) donor blood, C) control blood, D) HGSOC blood, and E) HGSOC ascites.

FIG. 9. Naïve, central memory, and effector memory T cells were flow sorted from healthy donor blood. Gating strategy for T cells: naïve (CD3⁺CD45RA⁺RO^{neg}CD62L⁺), central memory (CD3⁺CD45RA^{neg}RO⁺CD62L⁺), and effector memory (CD3⁺CD45RA^{neg}RO⁺CD62L^{neg}) populations.

FIG. 10. PMN-mediated T cell suppression requires PMN and ascites at time of initial T cell stimulation. T cells (CD3⁺) and PMN were used in autologous coculture at 1:1. PMN and/or ascites supernatants (ASC; 50% final well volume) were added to anti-CD3/CD28-stimulated T cells. After 72 h of coculture, T cell proliferation was measured by [³H] thymidine incorporation (16-18 h). The coculture experiments described were modified. A) T cells stimulated with anti-CD3/CD28 beads for 18 h prior to PMN and ASC addition are unable to be suppressed (n=3). B) T cells stimulated with anti-CD3/CD28 for 1-6 h prior to PMN and ASC addition are unable to be suppressed (n=2). C) T cells were cocultured with ASC and PMN for 5 min to 2 h prior to anti-CD3/CD28-stimulation. D-E) T cells were evaluated for surface expression of CD3 (D) and CD28 (E) after coculture with ASC and PMN for 1-4 h (n=3). F) PMN were treated with ASC or media. After 6 h, T cells and anti-CD3/CD28 beads were added to either pretreated PMN pellets or supernatants. Anti-CD3/CD28-stimulated T cell proliferation was not suppressed by PMN pellets or supernatants, as compared to the t=0 coculture (n=2). Symbols represent individual samples (n) and bars represent SEM. Statistical comparisons were by ANOVA with Tukey post-test (*, p<0.05; **, p<0.01; ***, p<0.001; ns, not significant). Results were consistent between CD4⁺ and CD8⁺ T cells.

FIG. 11. Ascites supernatants contain soluble proteins that induce the PMN suppressor phenotype. T cells (CD3⁺) and PMN were used in autologous coculture at 1:1. PMN and/or ascites supernatants (ASC; 50% final well volume) were added to anti-CD3/CD28-stimulated T cells. After 72 h of coculture, T cell proliferation was measured by [³H] thymidine incorporation (16-18 h). Prior to coculture, ASC were treated as follows. A) Proteinase K-digested ASC (PK-ASC; 100 μg/ml) abrogated the PMN suppressor phenotype (n=12). B) ASC were ultra-centrifuged at 200,000g for 1.5 h to fractionate membrane-associated proteins (200,000 g pellets termed membrane-rich, MR-ASC; membrane-depleted supernatants termed MD-ASC). MD-ASC retained the ability to induce the PMN suppressor phenotype, as compared to unmanipulated ASC, and MR-ASC induced the PMN suppressor phenotype to a lesser extent, as compared to unmanipulated ASC (n=7). Symbols represent individual samples (n) and bars represent SEM. Statistical comparisons were by ANOVA with Tukey post-test (*, p<0.05; **, p<0.01; ***, p<0.001; ns, not significant). Results were consistent between CD4⁺ and CD8⁺ T cells.

FIG. 12. Traditional pathways associated with granulocytic MDSC and N2 TAN do not play a role in the PMN suppressor phenotype. T cells (CD3⁺) and PMN were used in autologous coculture at 1:1. PMN and/or ascites supernatants (ASC; 50% final well volume) were added to anti-CD3/CD28-stimulated T cells. After 72 h of coculture, T cell proliferation was measured by [³H] thymidine incorporation (16-18 h). (A) L-arginine (50 μM-1 mM), N-acetylcysteine (NAC; 10-25 mM), or DNase I (50-100 IU) added to cocultures did not reverse ascites-induced PMN-mediated suppression. PMN pretreatment with CI-amidine (10-20 μM) for lh prior to coculture also did not abrogate the PMN suppressor phenotype (n=3). (B-D) Arachidonic acid metabolism does not play a role in the PMN suppressor phenotype. Indomethacin (INDO; 10 μM; n=5) (B), zileuton (ZLT; 50 μM; n=4) (C), or 1-methyl-DL-tryptophan (1-MT; 100 μM; n=4) (D) added to cocultures did not reverse the phenotype. (E) PMN treated with anti-TGF-beta receptor 1 (TGFbR1) Ab for lh prior to coculture had no effect on T cell proliferation. Treatment of PMN with IgG1 isotype (1-10 μg) similarly had no effect on proliferation (n=5). Symbols represent individual samples (n) and bars represent SEM. Statistical comparisons were by ANOVA with Tukey post-test (*, p<0.05; **, p<0.01; ***, p<0.001; ns, not significant). Results were consistent between CD4⁺ and CD8⁺ T cells.

FIG. 13. PMN suppressor phenotype inhibits upregulation of transcriptional factors responsible for effector cell differentiation. T cells (CD3⁺) and PMN were used in autologous coculture at 1:1. PMN and/or ascites supernatants (ASC; 50% final well volume) were added to anti-CD3/CD28-stimulated T cells. At 24, 48, 72, and 96 h of coculture, intracellular markers for transcriptional control by T-bet and Eomes were evaluated on CD8⁺ T cells. A) Gating strategy to delineate effector T cells (CD8⁺CCR7⁻ Eomes⁺T-bet^{hi}). B-G) Representative flow plots and quantification are shown for T cells that are B-C) unstimulated, D-E) stimulated, and F-G) stimulated with ASC and PMN (n=3). Symbols represent individual samples (n).

FIG. 14. Inflammation and injury, whether resulting from the tumor microenvironment or other pathologic conditions, can induce the PMN suppressor phenotype. A-F) Post-operative drainage fluid was collected 1d after debulking surgery for EOC. A) Gating strategy. B-D) Representative raw data from each post-operative drainage fluid that had sufficient cells for analysis. E) Post-operative drainage fluid is composed primarily of PMN (CD11b⁺CD33^{mid}CD15⁺CD14^{neg}DR^{neg}) with minimal proportions of monocytes/macrophages (CD11b⁺CD33^{hi}CD15^{neg}gCD14⁺DR⁺) (n=3). F) Paired ASC or post-operative drainage supernatants (POF; 50% final well volume) were added to anti-CD3/CD28-stimulated donor T cells and/or autologous donor PMN. After 72 h of co-culture, T cell proliferation was measured by [³H] thymidine incorporation (16-18 h). ASC and POF equally induce the PMN suppressor phenotype (n=7). G) Ascites were collected from patients with cirrhosis and without cancer (n=3). 1/3 cirrhotic ascites supernatants (Cirr-ASC) induced the PMN suppressor phenotype. Statistical comparisons were by ANOVA with Tukey post-test (*, p<0.05; **, p<0.01; ***, p<0.001; ns, not significant).

FIG. 15. Inhibition of complement C3 activation abrogates the PMN suppressor phenotype induced by malignant effusions. A) PMN treated with anti-CD11b (CR3) for lh prior to coculture abrogated the suppressor phenotype while pre-treating T cell with anti-ICAM-1 had no effect (n=5 ASC). B) Peptide inhibitor of C3 activation Cp40 (Cp40-ASC) completely abrogated the PMN suppressor phenotype, while scramble peptide (SCR-ASC) had no effect. The abrogation effect of Cp40 was highly robust, and was observed consistently in at least 10 separate experiments and 15 different ascites samples. A concentration-titration study showed that 2.5-20 ∥M Cp40 was sufficient to abrogate the PMN suppressor phenotype. C) Cryopreserved or fresh TALs (from n=3 patients) were cocultured with autologous ASC and/or PMN from healthy donors. Similar to circulating lymphocytes, the PMN suppressor phenotype inhibited anti-CD3/CD28-stimulated proliferation of TALs, and suppression was fully abrogated by Cp40-ASC. D) Malignant pleural fluid (MPF) from patients with a number of metastatic cancers (n=3 lung, n=1 breast, n=1 pancreatic, n=1 ovarian, n=1 lymphoma) also induced the PMN suppressor phenotype, which was abrogated by Cp40-ASC. Statistics are by ANOVA as compared to +CD3/CD28+ ASC+PMN (*, p<0.05; **, p<0.01; ***, p<0.001).

FIG. 16. Complement activation in ASC results in C3b/iC3b deposition on PMN and mediates the PMN suppressor phenotype via the AP and classical pathways. A) Activation of complement in ASC principally occurs via the AP and classical pathways, though inter-patient variability was observed. ASC were assessed for complement activity by WIESLAB ELISA kits that detect formation of C5b-9. Percent (%) activity is in relation to the provided positive control (n=38 ASC, **, p=0.0015; ****, p<0.0001). B) Activation of the AP was significantly higher (***, p=0.0004) in suppressor ASC versus non-suppressor ASC. C) PMN and T cells were cocultured in media, ASC, Cp40-ASC, or SCR-ASC for 1.5 h. Deposition of iC3b/C3b (clone 3E7/C3b recognizes both C3b and iC3b) on PMN was increased with ASC versus media, while iC3b/C3b deposition on T cells was unaffected by ASC (not shown). iC3b/C3b binding to PMN was decreased by Cp40-ASC, and unaffected by SCR-ASC (n=3 ASC). D) Inhibition of properdin (a-Prop; clone 6E11A4; 100 82 g/ml), which stabilizes the AP, resulted in a 1-log₁ increase in stimulated T cell proliferation as compared to isotype (IgG1k). Inhibition of the classical pathway (SALO; peptide; 1 μM) had an intermediate effect.

FIG. 17. NADPH oxidase is required for the PMN suppressor phenotype. We received blood from patients (n=5) with X-linked chronic granulomatous disease (CGD; gp91^{phox}-deficient) and healthy donors via overnight shipment from Dr. Steven Holland, MD (NIH/NIAID). PMN and T cells were cocultured in ASC, and anti-CD3/CD28-stimulated T cell proliferation was assessed. While healthy donor PMN acquired a suppressor phenotype after ASC exposure, CGD PMN did not. Statistics are by ANOVA as compared to the respective+CD3/CD28 (ns, non-significant; ***, p<0.001).

### DETAILED DESCRIPTION

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

The disclosure includes administering all drugs, and all combinations of drugs described herein, and thus any single drug or combination of drugs described herein can be expressly excluded from the scope of this disclosure. The disclosure includes all time periods, temperatures, ranges thereof, and all reagents and combinations of reagents described herein.

The present disclosure relates generally to identification of, and treating individuals, who have cancer and also have immunosuppressive neutrophils. Aspects of the disclosure are demonstrated using samples obtained from ovarian cancer (OC) patients, but it is believed to be more generally applicable to any individual who has cancer and immunosuppressive neutrophils, as described further below.

With respect to EOC, it is the leading cause of death from gynecological malignancies in the United States. EOC is typically diagnosed at advanced stages, presenting with peritoneal metastases and ascites accumulation. The tumor microenvironment of EOC is comprised of immunological niches that influence tumor progression and response to therapy (1). There is growing recognition for ascites as a distinct part of the EOC tumor microenvironment that facilitates seeding of serosal surfaces, mediates resistance to chemotherapy, and impairs anti-tumor immunity (2). Ascites contains specific tumor-associated lymphocyte populations that are being explored for cellular therapy (3), as well as immunosuppressive myeloid cells (4, 5) and exosomes (6) that are obstacles to anti-tumor immunity. Additional studies demonstrated distinct proteomic, glycosylation, and metabolic profiles in ascites that can affect tumor cell biology (7-9). The presence and volume of ascites at diagnosis of advanced EOC were associated with worse progression-free survival (PFS) and overall survival (OS) (10-12). These findings point to both cellular and soluble constituents of ascites influencing metastasis, anti-tumor immune responses, and prognosis.

The critical role of T cell immunity in EOC was demonstrated by tumor-infiltrating T cells at diagnosis predicting better outcomes (13). Intraepithelial CD8⁺ T cell accumulation and a high CD8⁺-to-Treg ratio were associated with favorable prognosis (14), while increased accumulation of Treg was associated with worse outcomes (15). Tumor-infiltrating CD8⁺ T cells recognizing NY-ESO-1, a tumor antigen, had impaired effector functions, but inhibiting LAG-3 and PD-1 signaling augmented proliferation and cytokine production (16). However, anti-PD-1 and anti-PD-L1 inhibitors have been largely ineffective in patients with relapsed/refractory EOC, with overall responses of 15% or less (17, 18), raising the notion of other suppressive pathways in the tumor microenvironment as obstacles to immunotherapy.

Myeloid-derived suppressor cells (MDSC) are defined as immature myeloid cells that suppress T cell responses. They include myeloid progenitors and immature mononuclear cells and granulocytes (19). Because MDSC markers overlap with other cell populations, phenotyping combined with demonstration of T cell suppression is optimal for identification of MDSC (20). Advanced cancer is associated with a myeloid bias characterized by increased frequencies of circulating granulocyte-monocyte progenitors that are skewed towards differentiating into granulocytes (21). Tumor-derived factors, such as G-CSF, GM-CSF, and IL-6, drive this myeloid bias (22), and result in a circulating and tumor-infiltrating MDSC population that accelerates tumor progression by suppressing T cell responses and releasing factors (e.g., VEGF and matrix metalloproteinases) that promote metastasis. EOC is associated with the accumulation of suppressive myeloid cells that correlate with worse outcomes. Cui et al. (23) found that MDSC in EOC triggered acquisition of stem cell-like features in cancer cells and increased metastatic potential. Myeloid cell (CD33⁺) accumulation in EOC was also associated with worse outcomes. B7-H4-expressing macrophages impeded T cell responses and correlated with more rapid tumor progression (24). These findings point to suppressive myeloid cells in the tumor microenvironment of EOC as barriers to anti-tumor immunity.

The concept of a myeloid bias can also apply to mature PMN, which is supported by the results described herein. For example, in patients with EOC, the pretreatment circulating PMN count (25) and the PMN-to-lymphocyte ratio (26) correlated with poor outcomes. Tumor-associated PMN can be broadly divided into N1 (anti-tumorigenic) or N2 (suppressive and pro-tumorigenic) populations, with distinct transcriptional profiles and functional properties (27). In addition, activated PMN can acquire a suppressive phenotype (28-30). Prior to the present disclosure, there was a gap in knowledge regarding the role of mature PMN as suppressor cells in the tumor microenvironment, as well as mechanisms for how PMN acquire the suppressor phenotype. The distinction between mature and immature suppressive granulocytes is mechanistically and therapeutically important. If circulating PMN are mature and acquire a suppressor phenotype within the tumor microenvironment as supported by the data presented herein, then therapeutic approaches such as those that are encompassed by this disclosure should focus on disabling their recruitment to the tumor microenvironment and target pathways driving suppression rather than approaches aimed at modulating myeloid programming in the marrow. Accordingly, in this disclosure, it is demonstrated that mature neutrophils are the suppressor granulocyte population in the ascites of patients with newly diagnosed EOC, and in other types of cancer. In particular, circulating neutrophils from these patients were not suppressive, but acquired a suppressor phenotype after ascites supernatant exposure. Ascites supernatants induced the same suppressor phenotype in normal donor neutrophils. Targeting of multiple neutrophil effector functions, including protein synthesis, exocytosis, vesicular trafficking, and complement C3 signaling, abrogated the suppressor phenotype. Thus, data presented herein provides advances in understanding of mature neutrophils as suppressor cells in the tumor microenvironment and also provides new approaches for therapeutic modulation to abrogate this barrier to anti-tumor immunity. In particular, this distinction between mature versus immature suppressive granulocytic cell population is significant in the context of rational design of therapeutic approaches to reverse this suppressive phenotype.

Furthermore, while the presence of an immunosuppressive factor in ascites from ovarian cancer patients has been previously recognized (see, for example, Bains et al., Gynecology and Obstetrics, (2016), Vol. 6, Issue 8, ISSN:2161-0932; Marotti, T. et al, Oncology (1982); 39:298-303) the present disclosure is believed to be the first to show that the immunosuppressive factors includes proteins and pathways that convert normal neutrophils into an immunosuppressive phenotype. This recognition accordingly provides for improved approaches to cancer care and diagnosis by exploiting this newly discovered nexus between certain proteins and pathways that affect formation of suppressive neutrophils.

Accordingly, the disclosure includes in various embodiments provides methods for determining whether or not an individual is a candidate for receiving a therapy described herein, and can further comprise administering the therapy to the individual. Determining whether or not an individual is a candidate for the therapy in one approach comprises exposing a biological sample from a cancer patient to normal neutrophils, and subsequently exposing the neutrophils to T cells, and measuring activation of T the cells, where reduced activation of the T cells relative to a control comprises the positive result and an indication that the individual has the immunosuppressive neutrophils. In an alternative embodiment, neutrophils obtained from the cancer patient can also be tested for an immunosuppressive characteristic, as further described below.

In a non-limiting approach, determination of immunosuppressive neutrophils can be performed as follows: (i) A sample comprising ascites supernatant from a patient is mixed with PMN and T cells from a healthy donor. (ii) T cells are stimulated with anti-CD3/anti-CD28 or another standard method for T cell activation. (iii) T cell activation is assessed by proliferation and expression of activation markers. (iv) The following controls may be used (a) unstimulated T cells as a negative control; (b) T cells stimulated with anti-CD3/anti-CD28 (positive control); (c) T cells cocultured with PMN and stimulated with anti-CD3/anti-CD28 (specificity control); (d) T cells cocultured with ascites supernatants and stimulated with anti-CD3/anti-CD28 (specificity control); (e) T cells cocultured with PMN and ascites supernatants and stimulated with anti-CD3/anti-CD28 (test condition). A positive result for ascites inducing a suppressor phenotype in PMN in embodiments comprises at least 1-log-fold reduction in T cell proliferation in condition € as compared to conditions (b), (c), and (d). The same approach can be applied to other malignant effusions, such as pleural effusions in lung cancer and mesothelioma, and other cancers. A positive result indicates that the malignant fluid renders PMN suppressive, and can be used as a basis for selection of patients for therapeutic approaches aimed at reversing or abrogating the PMN suppressor phenotype. The use of PMN from healthy donors in these assays means that the neutrophils are by definition mature and are not intrinsically skewed to an N2 phenotype, defined as circulating or tumor-associated PMN that are induced by factors in the tumor microenvironment (notably TGF-beta) to become suppressive. One advantage of using PMN and T cells from healthy donors is that the capacity for ascites and other malignant effusions to induce a suppressor phenotype in PMN can be tested with peripheral blood from healthy donors, which is widely available and easily standardized, and avoids potential problems of heterogeneity when using circulating white cells from patients with cancer.

Normal PMN can be obtained using any suitable approach. In embodiments, normal PMN are obtained from a donor who does not have cancer, and thus may comprise heterologous PMN. Thus, it will be recognized that suppressive PMN can be identified by a capability to suppress CD3/CD28-stimulated T cell proliferation, relative to a control, using any of a variety of approaches that will be apparent to those skilled in the art, given the benefit of the present disclosure. In embodiments T cell proliferation is assayed by measuring incorporation of a detectably labeled nucleotide into chromosomal DNA. Other T cell functions can also be measured using techniques that will be apparent to those skilled in the art.

Suitable controls can comprise any value obtained or derived from, for example, one or more T cell proliferation assays, and may include a known value or range of values, or may be a value or range of values determined from analysis of samples from a cohort of subject. Cohorts of subjects can be used to determine any value for normal PMN. Likewise, a cohort of individuals with cancer who have immunosuppressive PMN as described herein can be used to establish a control value.

In addition to T cell proliferation, other T cell responses that are established in the field include, but are not limited to, T cell activation markers, exhaustion markers, memory and effector phenotypes, antigen recognition, and lysis of target cells. In embodiments, the reference comprises a statistical value, such as an area under a curve, or another area or plot on a graph, obtained from repeated measurements of T cell proliferation, or a suitable alternative. The invention can also include determination of the effect of the biological sample from the individual on other cell types, including but not limited to other leukocytes. In one approach, the effect of the sample on PMN exocytosis is evaluated. The effect of the biological sample on other PMN functions, including but not limited to, reactive oxidant generation, phagocytosis, expression of activation and other surface markers, release of PMN constituents, and alterations in mRNA and protein expression, can be assessed by methods established in the field, including ELISA, Western blot. Q-PCR, and proteomics.

In embodiments, a separate but related approach is to test whether the patient's PMN are suppressive. In contrast to the embodiments above using autologous normal PMN and T cells, in this embodiment, PMN and T cells are collected from patients. Using the same general approach described above, the ability of circulating and tumor-associated PMN to suppress stimulated T cell proliferation and activation can be assessed. The results in patients with newly diagnosed EOC demonstrate the utility of this approach. In particular, it is demonstrated that while circulating PMN from patients with metastatic EOC were not intrinsically suppressive, they acquired a suppressor phenotype when mixed with ascites supernatants. Additionally, PMN purified from the ascites of EOC patients suppressed T cell proliferation. Similar approaches can be applied to malignant ascites and other effusions to delineate PMN accumulation in these sites by standard flow cytometry and/or CBC with differential, and whether they suppress stimulated T cell proliferation.

In embodiments, the individual tested and/or treated according to this disclosure has any cancer that is or may be affected by formation of immunosuppressive PMN. In embodiments, the individual has a cancer that causes effusions, such as ascites and pleural effusions. In embodiments, the gastrointestinal (GI) cancer comprises a cancer of any part of the GI tract, and/or accessory organs of the GI tract, which include but are not necessarily limited to malignancies located in the esophagus, stomach, biliary system, pancreas, small intestine, large intestine, rectum or anus. In embodiments, the individual has a cancer that produces a pleural effusion, such as any form of lung cancer, or mesothelioma. In embodiments, the individual has ovarian cancer. But as noted above, aspects of the present disclosure are demonstrated using lung, breast, and pancreatic cancer samples.

The biological sample that is exposed to normal PMN can be used directly, or can be processed using any suitable processing step to, for example, isolate, or concentrate components of the sample, including but not necessarily limited to proteins that may be present in the biological sample. In embodiments, the biological sample is a liquid biological sample. In embodiments, the biological sample is a cell-free liquid biological sample. In embodiments, the sample comprises a pleural effusion, or ascites, or a supernatant obtained from processing a sample of ascites.

Upon a determination that the individual has immunosuppressive PMN as described above, the disclosure can include administering to the individual an agent that reverses the immunosuppressive character of the PMN, and/or inhibits formation of the immunosuppressive PMN. In embodiments, the agent comprises an inhibitor of complement C3 signaling, or an inhibitor of downstream complement components. In embodiments, the agent comprises a drug that inhibits exocytosis, such as exocytosis that participates in vesicular trafficking, such as SNARE-dependent exocytosis. In embodiments the drug is an inhibitor of nicotinamide adenine dinucleotide phosphate-oxidase (NADPH oxidase).

In embodiments, an inhibitor of complement C3 signaling comprises a peptide that selectively binds to native C3. Alternatively, an inhibitor of one or more proteins of downstream pathways of complement C3, such as C3b, iC3b, can be used. In embodiments, the C3 inhibitor comprises compstatin, the compstatin derivative Cp40, PEGylated Cp40, or AMY-101, or a combination thereof. Other suitable complement inhibitors are known in the art and are encompassed by this disclosure.

In embodiments, the agent that inhibits exocytosis, such as SNARE-dependent exocytosis, comprises a SNARE decoy inhibitor. In embodiments, the drug is a fusion protein containing the TAT cell permeability sequence and either the SNARE domain of syntaxin-4 or the N-terminal of SNAP23. In embodiments, the agent is a drug that blocks SNARE protein activity in PMN.

In embodiments, the agent that inhibits NADPH oxidase comprises a reactive oxygen species (ROS) scavenger, such as or small molecule inhibitors of NADPH oxidase.

In embodiments, a drug is administered to a cancer patient in a therapeutically effective amount. Therapeutically effective amounts of certain drugs described herein have already been established in the art. For any drug described herein where a therapeutically effective amount is not established, the therapeutically effective amount, e.g., a dose, can be estimated initially either in cell culture assays or in animal models. Such information can then be used to determine useful doses and routes for administration in humans. A precise dosage can be selected by the individual physician in view of the patient to be treated. Dosage and administration can be adjusted to provide sufficient levels of the active moiety or to maintain the desired effect. Additional factors which may be taken into account include the severity and type of the disease state, age, weight and gender of the patient, desired duration of treatment, method of administration, time and frequency of administration, drug combination(s), reaction sensitivities, and tolerance/response to therapy. A therapeutically effective amount is an amount that reduces one or more signs or symptoms of a disease, and/or reduces the severity of the disease. A therapeutically effective amount may also inhibit or prevent the onset of a disease, or a disease relapse. In embodiments, a therapeutically effective amount inhibits growth of a tumor, and/or inhibits metastasis, and/or eradicates cancer from the individual. The drug(s) can be administered to an individual using any suitable route of administration, including but not necessarily limited to via intravenous, intraperitoneal, subcutaneous, intra-articular, or oral administrations, depending on the particular drug and cancer being treated. The drugs may be introduced as a single administration or as multiple administrations or may be introduced in a continuous manner over a period of time. For example, the administration(s) can be a pre-specified number of administrations or daily, weekly or monthly administrations, which may be continuous or intermittent, as may be therapeutically indicated.

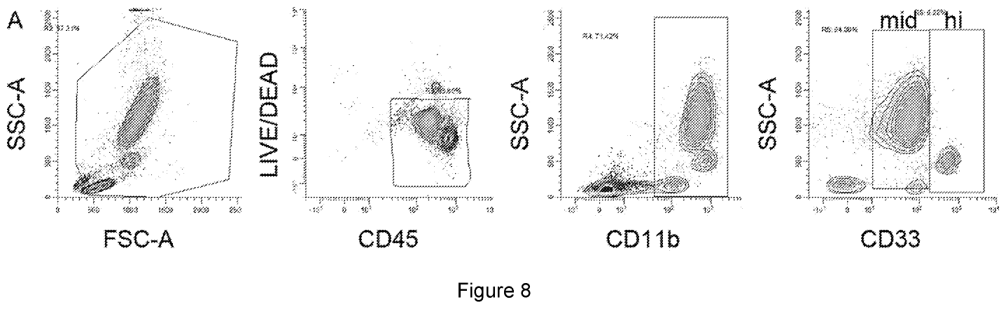

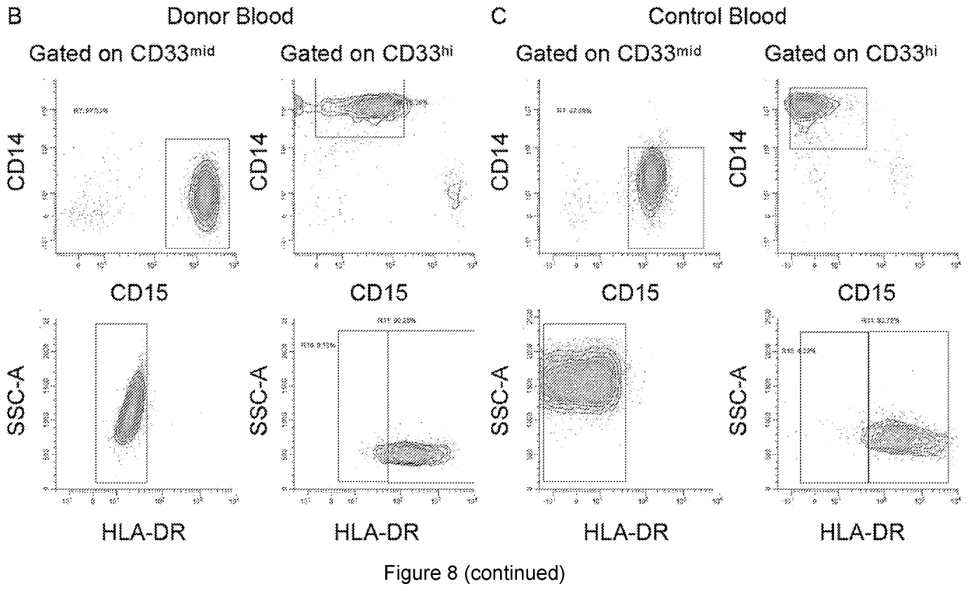

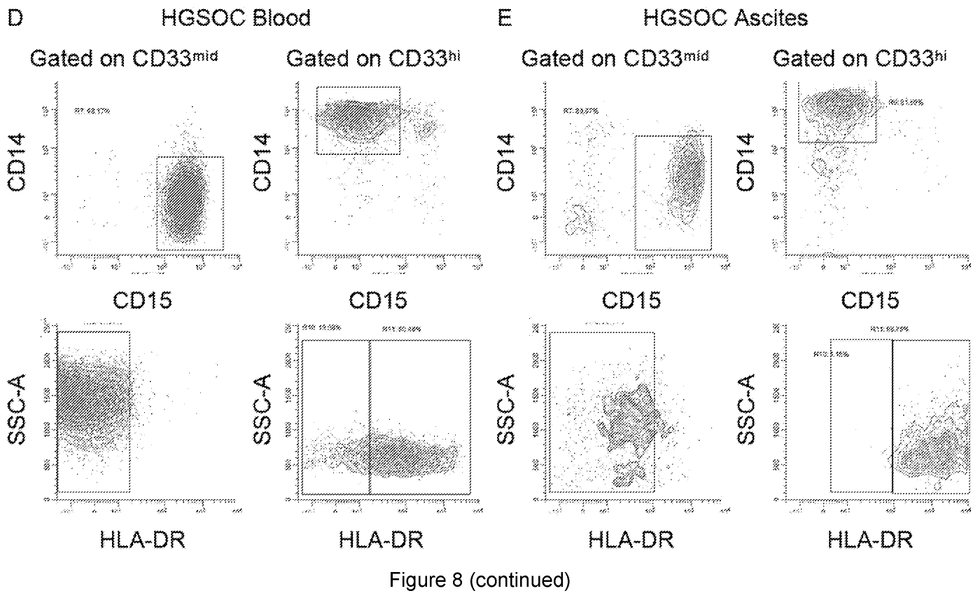

Thus, by reversing, inhibiting or preventing PMN from adopting an immunosuppressive phenotype the present invention provides for sensitizing ovarian cancer and other cancers to immune checkpoint inhibitors, many of which are presently known in the art, some of which are currently undergoing human trials, and others which are already in use. In this regard, an example of an immune checkpoint inhibitor target is a transmembrane programmed cell death (PD) protein. In certain embodiments, the checkpoint inhibitors that are combined with an inhibitor comprise antibodies that bind to PD-1 (e.g., Pembrolizumab and Nivolumab) or anti-PD-L1 (e.g., Avelumab). In another embodiment, the checkpoint inhibitor is an antibody that targets CTLA-4, such as Ipilimumab. In another embodiment the checkpoint inhibitor targets CD366 (Tim-3), which is a transmembrane protein also known as T cell immunoglobulin and mucin domain containing protein-3. The disclosure thus includes administering to an individual in need thereof an agent capable of inhibiting the immunosuppression of PMN, and also administering to the individual an anti-cancer agent, such as an immune checkpoint inhibitor, wherein the efficacy of the anti-cancer agent is increased relative to its function in the absence of inhibition of the immunosuppression. In certain implementations, the individual has been previously treated for cancer with a checkpoint inhibitor, and the cancer was initially resistant, or develops resistance, to the checkpoint inhibitor treatment. The disclosure thus includes selecting an individual who has a cancer that is resistant to a checkpoint inhibitor, and administering to the individual an inhibitor of the PMN immunosuppressive phenotype and the checkpoint inhibitor. In certain embodiments a combination of an immune checkpoint inhibitor and an inhibitor of the PMN immunosuppressive phenotype may have a synergistic effect against cancer, which may comprise but is not limited to a greater than additive inhibition of cancer progression, and/or a greater than additive inhibition of an increase in tumor volume, and/or a reduction in tumor volume, and/or a reduction in tumor growth rate, and/or an eradication of a tumor and/or cancer cells. The method may also result in a prolonging of the survival of the individual. In addition to augmenting the anti-tumor capacity of checkpoint inhibitors, inhibition of the suppressive phenotype may augment the anti-tumor efficacy of other immunotherapies, which include, but are not limited to, anti-tumor vaccination and use of adoptive T cell therapy, such as CART cells and T cells with engineered T cell receptors. In support of this approach, ascites-stimulated PMN acquire a suppressor phenotype that inhibits the expansion of T cells with an engineered T cell receptor being developed for adoptive cell therapy for EOC] (FIG. 8).

In embodiments, the disclosure comprises identifying an individual as having immunosuppressive PMN as described herein, and administering a combination of a complement inhibitor and an immune checkpoint inhibitor, such as described in US patent publication no. 20170246298, from which the description of complement inhibitors, immune checkpoint inhibitors, and methods of administering such inhibitors is incorporated herein by reference. In embodiments, subsequent to identifying an individual as having immunosuppressive PMN as described herein, a drug that is described in U.S. Pat. Nos. 10,308,687; 10,125,171; 10,125,171; and 10,035,822, from which the description of drugs, sequence listings, methods of administration, and dosing, are encompassed herein by reference.

Another aspect of the disclosure comprises inhibition of PMN migration and/or functional properties related to their ability to suppress T cell responses. Drug formulation(s) that inhibits PMN trafficking (e.g. CXCR2 inhibitors) to the tumor microenvironment or degranulation or release of suppressive products are expected to reverse PMN suppression of T cell function. Therefore, use of such agents may enhance the benefit of existing and investigational immunotherapies, including, but not limited to, checkpoint inhibitors, vaccination, and adoptive T cell transfer.

The following examples are intended to illustrate, but not limit the invention.

### EXAMPLE 1

This example demonstrates that ovarian cancer ascites induce patient circulating PMN to become T cell suppressive.

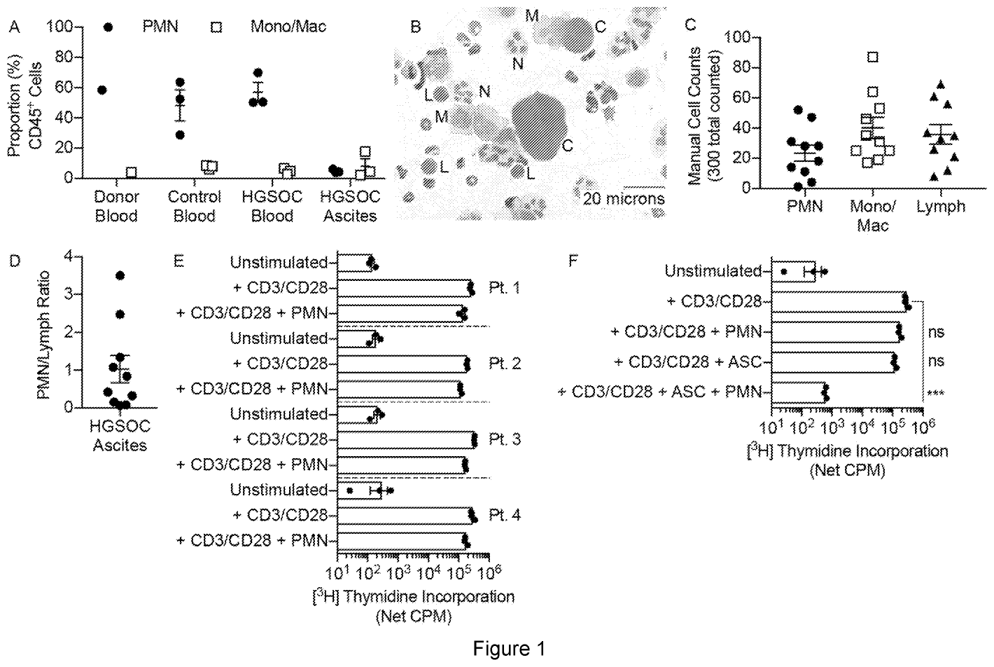

Ascites from patients with newly diagnosed EOC contains monocytes/macrophages and granulocytes with variable immunosuppressive phenotypes (4, 5). Since MDSC are defined as immature, we compared the major populations of circulating and ascites WBC and their maturity based on standard cytologic criteria. In routine pre-operative CBC testing, patients with newly diagnosed EOC had normal circulating WBC numbers and differentials. Granulocytes were >99% mature segmented PMN, bands were <1%, and no immature granulocytes were observed (Table 1). We found no difference in the proportions of circulating PMN (CD45⁺CD11b⁺CD33^{mid}CD15⁺CD14^{neg}DR^{neg}) and monocytes (CD45⁺CD11b⁺CD33^{hi}CD15^{neg}CD14⁺DR⁺) between patients with high-grade serous ovarian cancer (HGSOC; accounts for majority of all cases) and female patients undergoing surgery for a benign adnexal mass (control blood) (FIG. 8). A hematopathologist (JTW) analyzed the cellular composition and morphology of granulocytes in Wright Giemsa-stained cytospins of ascites from patients with newly diagnosed metastatic HGSOC (FIG. 1B-C). The granulocytes had segmented nuclei with prominent filaments characteristic of mature PMN. No immature granulocytes were observed. The ascites PMN-to-lymphocyte ratio was 1.03 [95% CI 0.21-1.8, SEM 0.4] (FIG. 1D). These results demonstrate that the inflammatory microenvironment in ascites is distinct from blood, and circulating and ascites PMN are morphologically mature.

Because we previously observed that ascites granulocytes suppressed stimulated T cell proliferation (5), we evaluated whether circulating PMN from patients with advanced EOC were suppressive. We assessed the proliferation of anti-CD3/CD28-stimulated T cells from patients with newly diagnosed EOC (n=4) after incubation with media, autologous PMN, and/or ascites supernatants. The coculture PMN-to-lymphocyte ratio was 1:1, corresponding to the mean ratio observed in ascites. Addition of either PMN or ascites alone resulted in negligible reductions in stimulated T cell proliferation (FIG. 1E and F). However, when added together, the interaction effect of PMN and ascites reduced T cell proliferation by a factor of 2.08 log₁₀ [95% CI 1.26-2.90, p=0.0002] (FIG. 1F). These results establish that ascites induce mature PMN to acquire a suppressor phenotype, and are consistent with the concept that mature, circulating PMN acquire this suppressor phenotype upon recruitment to the TME.

### EXAMPLE 2

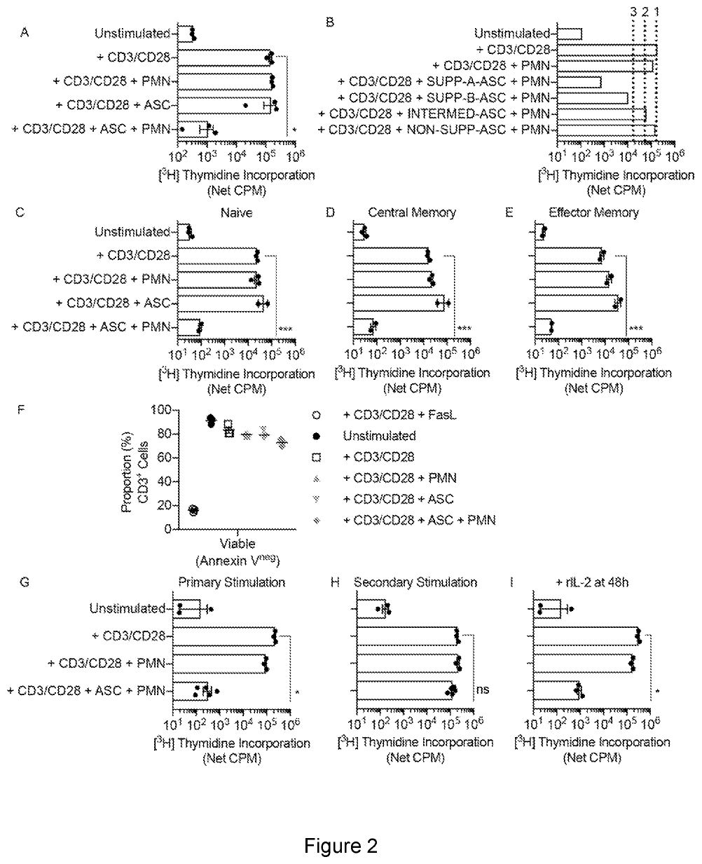

This example demonstrates that ovarian cancer ascites induce circulating PMN from healthy donors to acquire the suppressor phenotype. In patients with metastatic EOC, it is possible that tumor-derived factors could influence marrow and circulating granulocytes to render them more sensitive to the effects of ascites. We recently showed that ascites rendered PMN from healthy donors T cell suppressive (30). In the current study, we extended these results to include a larger number of EOC ascites and histology other than HGSOC (n=31; Table 2). PMN and T cells from a cohort of healthy donors were used for each experiment. Similar to patient PMN, ascites rendered PMN suppressive when cocultured with autologous T cells stimulated with anti-CD3/CD28 microbeads and soluble anti-CD3/CD28 Ab (FIG. 2A). Again, addition of PMN or ascites alone resulted in small biological effects (0.21 and 0.24 log₁₀ reductions).

We stratified ascites (n=31) into three categories based on the induction of a PMN suppressor phenotype, where x equals a reduction in proliferation as compared to anti-CD3/CD28-stimulated T cells alone: suppressors (x≥1 log₁₀), intermediate suppressors (0.5 log₁₀≤x<1 log₁₀), and non-suppressors (x<0.5 log₁₀) (FIG. 2B, Table 2). These results include ascites from n=22 HGSOC patients reported in our recent study (30). From this point on, we pre-selected ascites known to induce the PMN suppressor phenotype (x≥1 log₁₀) in order to evaluate mechanisms for PMN-mediated suppression. Together, these findings show that mature PMN are the suppressive granulocytic population in EOC ascites, and are rendered suppressive by factors in the TME.

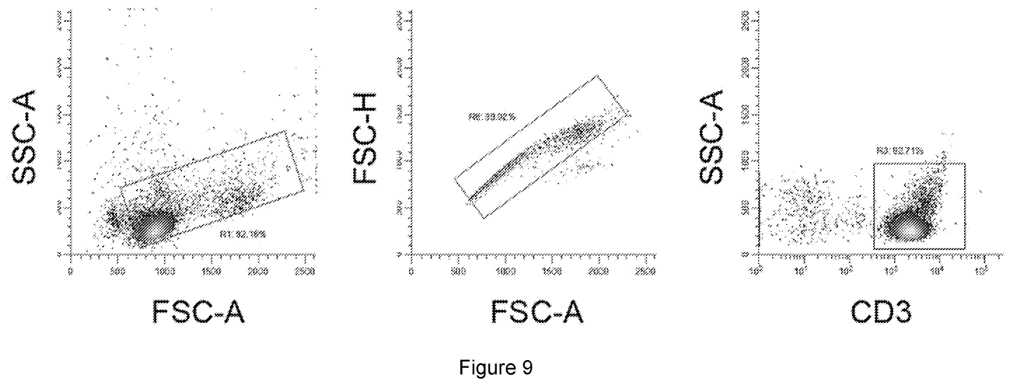

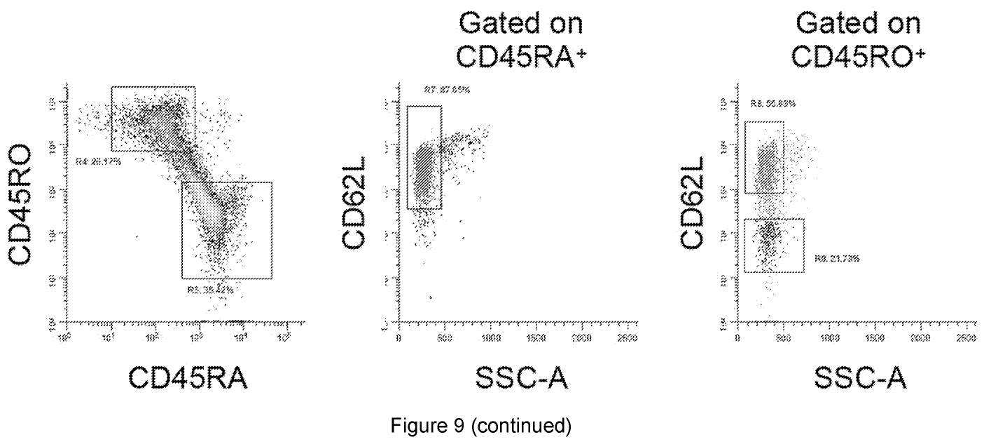

An effective anti-tumor response requires expansion and activation of tumor antigen-specific effector T cells in the TME. To determine whether the PMN suppressor phenotype affected central and effector memory T cells, we isolated naïve (CD3⁺CD45RA⁺RO^{neg}CD62L⁺), central memory (CD3⁺CD45RA^{neg}RO⁺CD62L⁺), and effector memory (CD3⁺CD45RA^{neg}RO⁺CD62L^{neg}) T cell populations from blood (FIG. 9). All anti-CD3/CD28-stimulated T cell populations were suppressed by the PMN suppressor phenotype (FIG. 2C-E). Since the PMN suppressor phenotype had a similar effect on naïve, central memory, and effector memory T cells, we used unfractionated T cells in subsequent experiments. These findings showing that the PMN suppressor phenotype acts on the major circulating T cell populations, including effector memory T cells that drive anti-tumor immunity, support the importance of suppressive PMN as obstacles to anti-tumor immunity.

### EXAMPLE 3

This example demonstrates that suppressed T cells are viable and suppression is reversible.

We asked whether the observed reduction in stimulated T cell proliferation when cocultured with PMN and ascites was due to T cell apoptosis. The proportion of apoptotic stimulated T cells cocultured with media, ascites supernatants and/or PMN ranged from 17-27% (FIG. 2F). In addition, when T cells were cocultured with ascites and PMN, T cell proliferation was restored with ascites removal and anti-CD3/CD28 re-stimulation (FIG. 2G and H). Addition of recombinant IL-2 (rIL-2) to cocultures at 48 h did not reverse T cell suppression (FIG. 21). These results argue against T cell apoptosis as a mechanism for the PMN suppressor phenotype and show the potential for reversibility of T cell suppression.

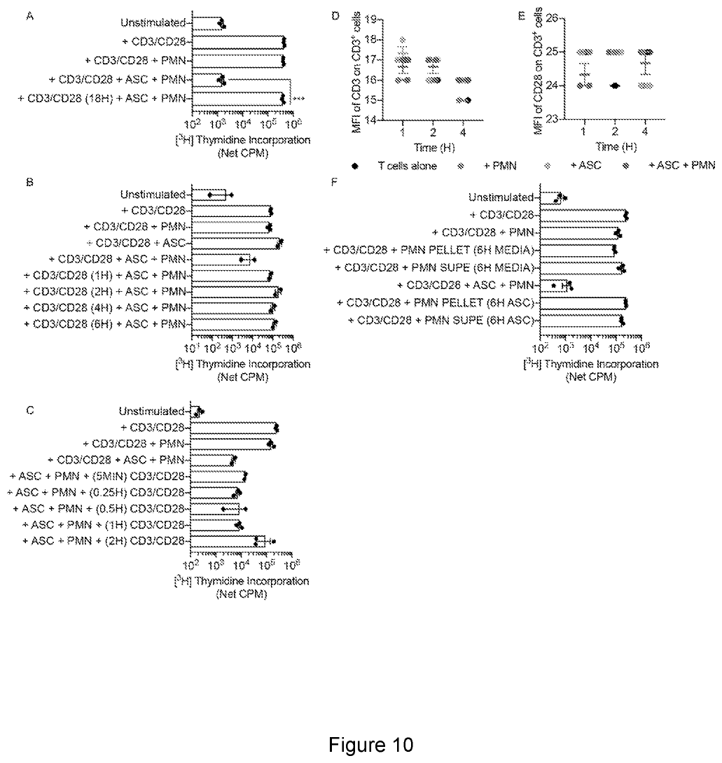

Next, we carried out a series of experiments to identify the time frame of T cell suppression in relation to anti-CD3/CD28-stimulation and exposure to PMN and ascites. When T cells were anti-CD3/CD28-stimulated for 18 h (FIG. 10A) or 1-6 h (FIG. 10B) and then cocultured with ascites and PMN, T cell proliferation was unimpaired. However, when T cells were cocultured with ascites and PMN followed by addition of anti-CD3/CD28-stimulation at various time points, suppression of T cell proliferation occurred when anti-CD3/CD28 was added within lh of coculture, but was lost at 2 h (FIG. 10C). Surface expression of CD3 and CD28 on T cells after incubation with ascites and/or PMN was similar to T cells incubated with media (FIG. 10D), indicating that the mechanism for T cell suppression is not due to loss of CD3 and CD28. These results show that the PMN suppressor phenotype requires PMN and ascites exposure early after T cell stimulation, is reversible, and raises the potential for therapeutically abrogating the suppressor phenotype.

### EXAMPLE 4

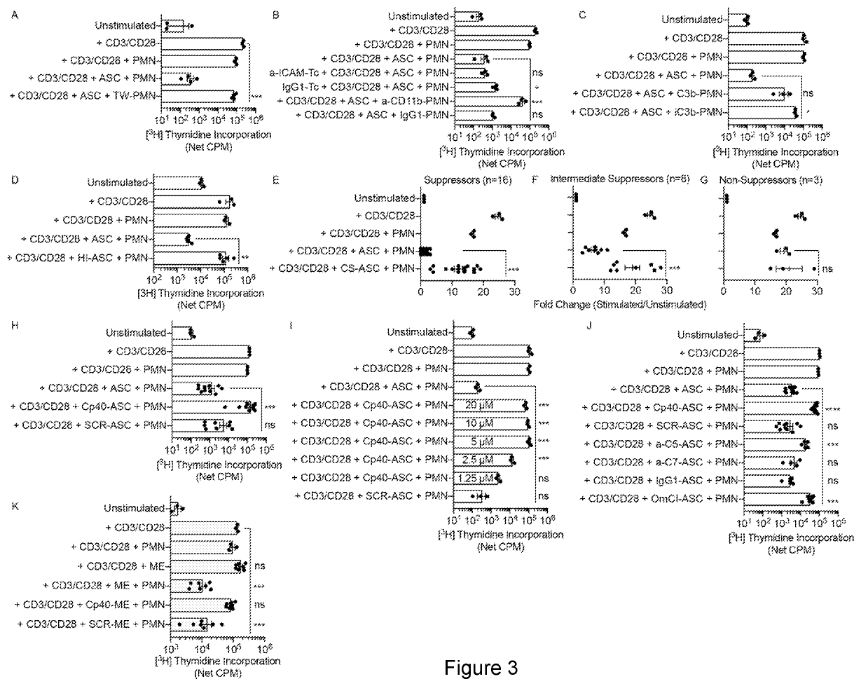

This example demonstrates that PMN suppressor phenotype requires T cell contact and complement C3 activation. We previously observed that ascites stimulated PMN degranulation and the generation of PMN extracellular traps (NETs) (30), raising the possibility that soluble products may be released into the coculture and mediate suppression of T cells. In the current study, when we exposed PMN to ascites for 6 h and then added the PMN pellets or supernatants to anti-CD3/CD28-stimulated T cells, proliferation was unimpaired (FIG. 10E). In addition, separation of PMN and T cells using a transwell system resulted in abrogation of suppression (FIG. 3A), suggesting that cell contact between PMN and T cells is required for suppression.

Complement receptor 3 (CR3; Mac-1; CD11b/CD18) mediates a critical step in PMN recruitment and cell-cell adhesion by binding to ICAM-1 on endothelial and T cells. Pretreating PMN with anti-CD11b Ab abrogated suppression, while pretreating T cells with anti-ICAM-1 Ab had no effect (FIG. 3B). Pretreating PMN or T cells with IgG1 as an isotype control had no effect. In humans, endotoxin (LPS) challenge or severe injury resulted in a subset of circulating PMN (CD11c^{bright}CD62L^{dim}CD11b^{bright}CD16^{bright}) that mediated T cell suppression through oxidant generation and CD11b (31). We observed that after 24 h, PMN in media or ascites variably downregulated CD62L and CD16 expression, but there was no discernible population with increased CD16 expression as compared to baseline (data not shown), suggesting that the PMN suppressor phenotype induced by ascites is distinct from circulating PMN suppressors induced by acute systemic inflammation. CR3 also binds iC3b, a cleavage product of C3 that acts as an opsonin and mediates intracellular signaling. Pretreating PMN with either C3b or iC3b prior to coculture resulted in abrogation of the suppressor phenotype, suggesting a desensitizing effect on PMN (FIG. 3C).

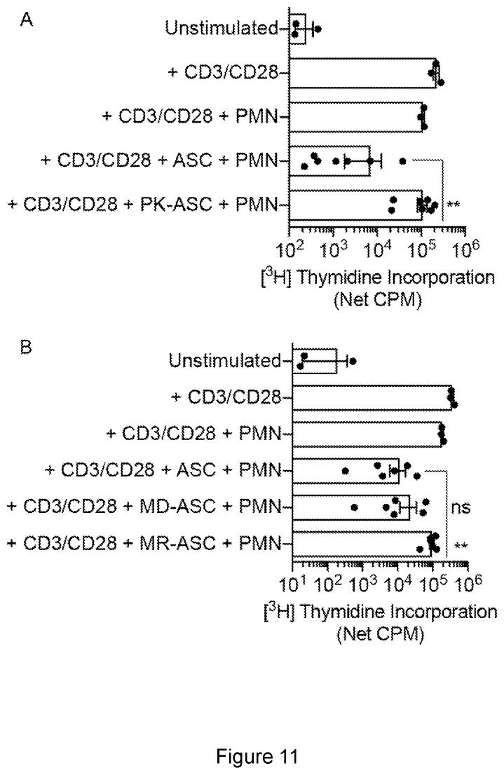

To determine if the factor in ascites inducing the PMN suppressor phenotype was complement-related, we first evaluated if it was a heat-labile protein(s) via heat-inactivation (HI-ASC; FIG. 3D) and proteinase-K digestion (PK-ASC FIG. 11A) of ascites supernatants prior to addition to cocultures. Both treatments completely abrogated T cell suppression. Since ascites exosomes can inhibit T cell responses (6), we determined whether the suppressive factor was membrane-associated or soluble. The ascites were ultracentrifuged to separate the membrane-rich (MR-ASC) and membrane-deplete (MD-ASC) fractions. The MR-ASC neither suppressed T cell proliferation alone nor in combination with PMN, while the MD-ASC rendered PMN suppressive (FIG. 11B). These results show that soluble, heat-labile protein(s) in ascites are required for the PMN suppressor phenotype.

C3 plays a central role in the activation of the three complement pathways: classical, alternative, and lectin. Compstatins are a family of peptides that inhibit complement activation by binding to native C3 and interfering with convertase formation and C3 cleavage, and are being developed as therapeutics for complement-driven disorders (32, 33). To test the role of C3 activation in the PMN suppressor phenotype, we treated ascites with compstatin (CS-ASC; 250 μM; n=27) (FIG. 3E-G) and Cp40 (Cp40-ASC; 20 μM; n=10) (FIG. 3H) prior to coculture with PMN and T cells. Both completely abrogated the PMN suppressor phenotype, while scramble peptide (SCR-ASC) had no effect. The mean PMN viability (based on PI^{neg}Annexin-V^{neg}) after 24 h exposure to Cp40-ASC (n=4) was 68±8%, which was similar to untreated ascites (80±8%) and SCR-ASC (80±10%) (p=ns) (data not shown). PMN viability ranged between 41-47% at 54 h under these conditions; these results do not support excess PMN death as a mechanism for Cp40 abrogating the PMN suppressor phenotype. A concentration-titration study showed that 5 μM Cp40 was sufficient to fully abrogate the PMN suppressor phenotype (FIG. 3I).

To evaluate the role of downstream complement proteins in mediating the PMN suppressor phenotype, ascites were pretreated with Ab against C5 or with OmCI, a peptide C5 inhibitor derived from the saliva of Ornithodoros moubata (34, 35), prior to coculture. Inhibiting C5, with either antibody or peptide, had a partial abrogating effect on T cell suppression, as compared to Cp40 that fully abrogated the PMN suppressor phenotype (FIG. 3J). The membrane attack complex (MAC; C5b-C9) disrupts membranes of target cells leading to cell lysis. Ab against C7, a required component of MAC, had no effect on T cell suppression. These results show that functional CR3 and activation of C3 are required, C5 has an intermediate effect, and MAC is unlikely to be involved in the PMN suppressor phenotype.

We analyzed whether the PMN suppressor phenotype induced by EOC ascites would also occur following PMN exposure to other malignant effusions. We observed a similar PMN suppressor phenotype when PMN and anti-CD3/CD28-stimulated T cells were cocultured with malignant pleural and ascites supernatants from patients with a number of metastatic cancers (18/20 samples induced a PMN suppressor or intermediate suppressor phenotype). Using samples that met the suppressor definition (Table 3), the PMN suppressor phenotype induced by malignant pleural and ascites was also abrogated by Cp40-treatment (FIG. 3K). These data demonstrate the generalizability of our findings regarding the C3-dependent induction of the PMN suppressor phenotype in malignant effusions.

### EXAMPLE 5

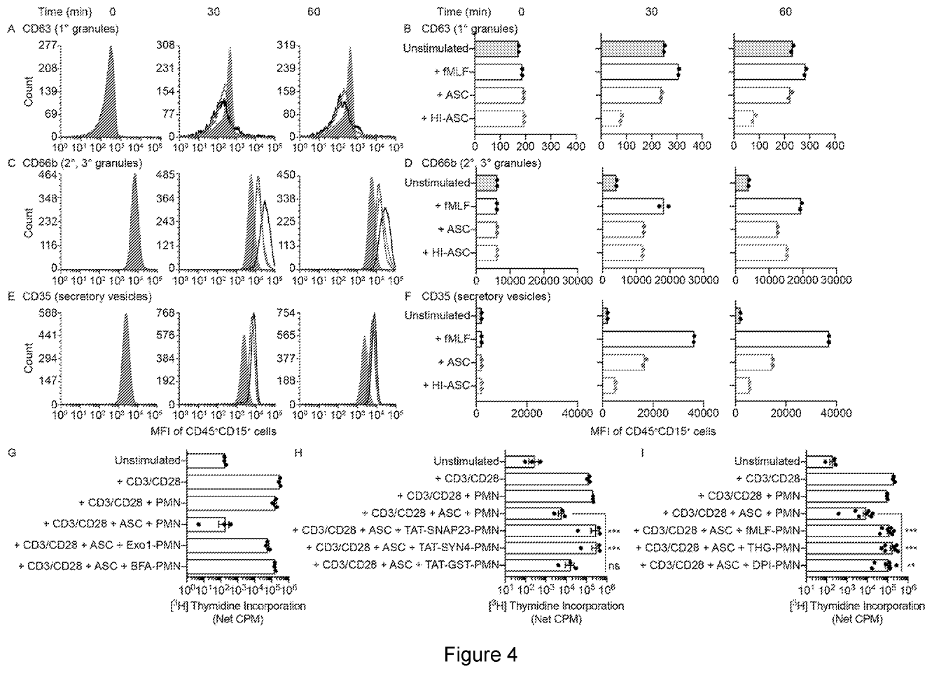

This example demonstrates that ascites activates multiple PMN effector pathways that mediate suppression. We undertook a comprehensive analysis of the role of effector pathways in mediating the PMN suppressor phenotype. Since PMN degranulation can result in the release of suppressive products, including arginase-1 (28), we evaluated PMN surface expression for markers of fusion of primary (CD63), secondary and tertiary (CD66b) granules, and secretory vesicles (CD35; complement receptor 1, CR1) after 30 and 60 min exposure to media, N-Formylmethionine-leucyl-phenylalanine (fMLF; positive control), ascites supernatants, or HI-ASC. CD63 surface expression was unaffected by ascites (FIG. 4A-B), and CD66b surface expression was no different between untreated ascites and HI-ASC (FIG. 4C-D). However, CD35/CR1 surface expression increased with ascites as compared to media, and decreased again with HI-ASC (FIG. 4E-F). These results suggest that ascites induce variable effects on fusion of PMN granules and secretory vesicles.

To assess the effect of endoplasmic reticulum (ER) transport on the PMN suppressor phenotype, we pretreated PMN with brefeldin-A and an ER export inhibitor, Exo1. Both agents abrogated the PMN suppressor phenotype (FIG. 4G). We further evaluated the role of exocytosis using fusion proteins containing the TAT cell permeability sequence and either the SNARE domain of syntaxin-4 or the N-terminal of SNAP23. SNARE decoys inhibit stimulated exocytosis of secretory vesicles and secondary and tertiary granules, but not primary granules, in PMN (36, 37). PMN pretreated with the SNARE decoys for SNAP23 and syntaxin-4 abrogated the PMN suppressor phenotype, while TAT fusion proteins with GST, a specificity control, had no effect (FIG. 4H). These results show that SNARE-dependent exocytosis is required for the PMN suppressor phenotype.

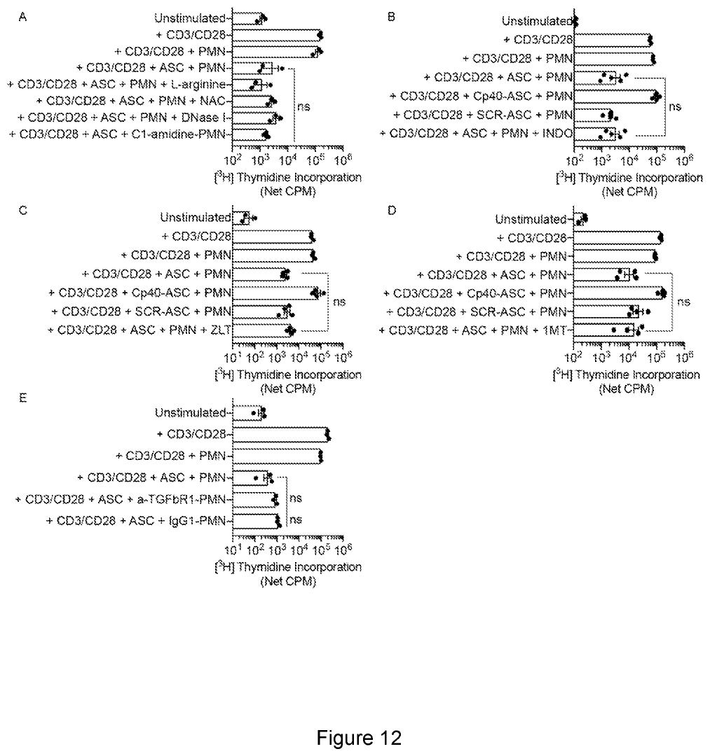

Activation with fMLF induced PMN to inhibit T cell responses through a mechanism requiring hydrogen peroxide generation (27). Therefore, we asked whether pretreatment of PMN prior to ascites exposure would desensitize PMN. We observed that activation with fMLF prevented induction of the PMN suppressor phenotype, indicative of heterologous desensitization (FIG. 4I). In addition, PMN pretreated with thapsigargin (THG; inhibitor of Ca²⁺ mobilization) abrogated the PMN suppressor phenotype. To determine the role of PMN ROS in suppressing T cell proliferation, we evaluated the effect of the ROS scavenger, N-acetyl cysteine (NAC), and diphenyleneiodonium (DPI), a small molecule flavocytochrome inhibitor of NADPH oxidase, the major source of PMN ROS generation. Addition of NAC to cocultures did not reverse suppression (FIG. 12A), while pretreating PMN with DPI did abrogate the PMN suppressor phenotype (FIG. 4I). These results are conflicting regarding the role of ROS generation in the PMN suppressor phenotype; further studies using PMN from patients with chronic granulomatous disease (CGD), an inherited disorder of the phagocyte NADPH oxidase, will delineate the role of NADPH oxidase in the PMN suppressor function.

Release of arginase-1 from tertiary granules can also suppress T cells (28). Addition of L-arginine to the cocultures had no effect on T cell suppression (FIG. 12A), arguing against arginase-1 mediating the PMN suppressor phenotype. We previously observed that ascites stimulated NET generation (30), and Lee et al. (38) recently demonstrated that NETs facilitated premetastatic niche formation in murine EOC. However, pretreatment of PMN with CI-amidine, an inhibitor of protein arginine deiminase 4 required for NET generation, or addition of DNase Ito the cocultures to degrade NETs, had no effect on the PMN suppressor phenotype (FIG. 12A), suggesting that the mechanism is independent of NETs. Wong et al. (39) recently showed that IFN-gamma and TNF-alpha synergize to induce cyclooxygenase-2 (COX2) in the EOC TME, which in turn hyperactivates MDSC and leads to overexpression of the immunosuppressive enzyme, indoleamine-2,3-dioxygenase (IDO). We observed that addition of indomethacin, a non-selective COX inhibitor (FIG. 12B), zileuton, an inhibitor of 5-lipoxygenase (FIG. 12C), and 1-methyl-DL-tryptophan, an inhibitor of IDO1/2 (FIG. 12D), to the cocultures had no effect on the PMN suppressor phenotype, indicating that the arachidonic acid pathway and

IDO are likely not playing a role. High levels of TGF-beta are present in EOC ascites (40), and TGF-beta signaling can skew TAN to a suppressive N2 phenotype (24). We observed that anti-TGF-beta receptor 1 Ab did not abrogate the PMN suppressor phenotype (FIG. 12E). Together, these results show that multiple PMN effector pathways are required for mediation of the suppressor phenotype in mature PMN that are distinct from those associated with MDSC or N2 TAN.

### EXAMPLE 6

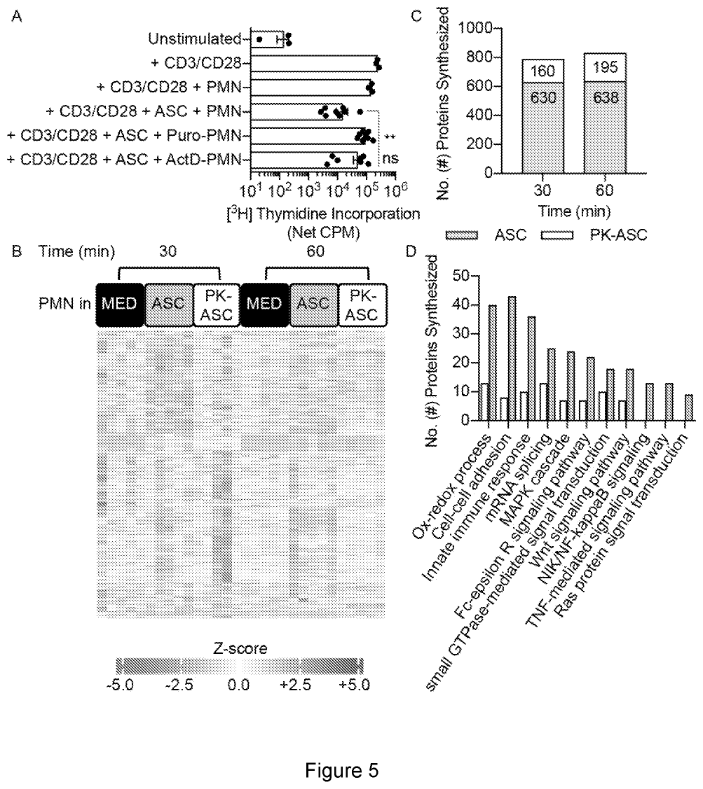

This example demonstrates that ascites induce robust protein synthesis in PMN that is required for the suppressor phenotype. We asked whether protein synthesis in PMN was required for the suppressor phenotype. Pretreating PMN with the protein synthesis inhibitors, puromycin and actinomycin D, resulted in variable abrogation of the suppressor phenotype (FIG. 5A). Based on these results, we explored the effect of ascites on protein production in PMN. We exposed PMN to media, ascites supernatants, or PK-ASC for 30- and 60-min, and subsequently underwent proteomics analysis. Unique protein groups (1,935) were quantified with >2 peptides per protein and <2% missing data rate on the protein level. Proteome patterns were similar at 30- and 60-min time points, and showed prominent discrepancies between PMN exposed to ascites versus PK-ASC (FIG. 5B). The PK-ASC-exposed PMN displayed a proteome pattern more similar to PMN exposed to media. Under the selected cutoff thresholds (>1.5-fold protein change, p<0.05) at 30- and 60-min, 630 and 638 proteins exhibited significant changes in the ascites groups, while only 160 and 195 proteins were significantly changed in the PK-ASC groups, respectively (FIG. 5C). Notably, 175 and 173 proteins were exclusively changed with 30- and 60-min ascites exposure, respectively. Gene ontology analysis of significant proteins showed enrichment of multiple classes of proteins with diverse biological functions in the ascites-exposed PMN (FIG. 5D). KEGG pathway analysis showed that the transcription factors, STAT3 and its target PU.1, were highly upregulated in PMN exposed to ascites, as compared to PMN exposed to media (p=0.01 and p=0.002, respectively). Ascites led to increased levels of several granular constituents, including myeloperoxidase (p=0.005), neutrophil elastase (p=0.0001), cathepsin G (p=0.001), defensin 1 (p=0.02), defensin 3 (p=0.001), lysozyme C (p=0.03), and MMP9 (p=0.04). In addition, ascites exposure led to increased levels of multiple complement pathway and signaling components, including C1r (p=0.003), C1q receptor (p=0.01), C3 (p<0.0001), C5 (p<0.0001), C9 (p<0.0001), properdin (p<0.001), factor B (p<0.0001), and factor D (p<0.0001), as well as CR1 (CD35,p<0.0001) and CR3 (CD18, p=0.005; CD11b, p<0.0001). Ascites also led to decreased levels of a smaller subset of proteins involved in protein folding, microtubule-based processes, and response to ROS, including gp91^{phox} (p=0.01), SOD (p<0.0001), COX2 (p<0.0001), NADPH-dependent carbonyl reductase (p<0.0001), and promyelocytic leukemia protein (PML, p=0.03). These results suggest that ascites induces synthesis of multiple classes of proteins in PMN, and protein synthesis is required for the PMN suppressor phenotype.

### EXAMPLE 7

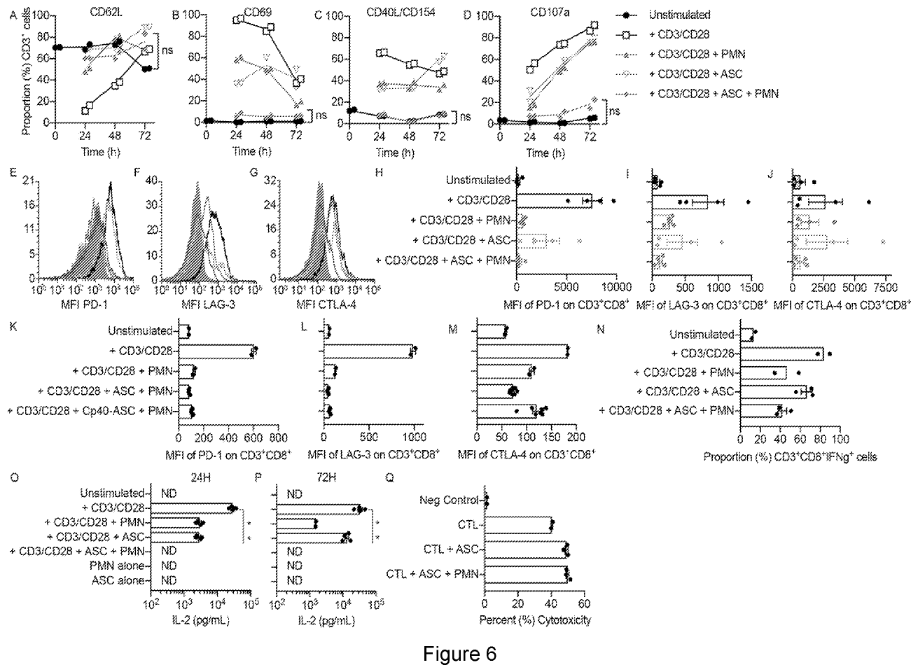

This example demonstrates that PMN suppressor phenotype inhibits stimulated naïve T cell activation without inducing exhaustion marker upregulation and without affecting antigen-specific CTL killing. To further delineate the effects of the PMN suppressor phenotype on T cell immunity, we evaluated markers for T cell activation and exhaustion in cocultures. The proportion of CD62L-expressing T cells decreased after 24 h of anti-CD3/CD28-stimulation as compared to baseline (characteristic of newly activated T cells), which was modulated by ascites or PMN alone, and inhibited in cocultures with ascites and PMN (FIG. 6A). The proportion of T cells expressing CD69 (FIG. 6B), CD40L (FIG. 6C), and CD107a (FIG. 6D) increased with stimulation as compared to baseline (characteristic of newly activated T cells); in each case, upregulation was inhibited in cocultures with ascites and PMN. In addition, anti-CD3/CD28-stimulation upregulated the expression of exhaustion markers PD-1, LAG-3, and CTLA-4 on T cells (FIG. 6E-J). Coculture with ascites or PMN alone had more variable effects, while the combination of ascites and PMN prevented anti-CD3/CD28-stimulated upregulation of PD-1, LAG-3, and CTLA-4. Finally, while Cp40-ASC abrogated the PMN suppressor phenotype and enabled robust anti-CD3/CD28-stimulated T cell proliferation, Cp40-ASC did not result in upregulation of PD-1 or LAG-3 on T cells (FIG. 6K-L), while CTLA-4 expression was upregulated relative to unstimulated T cells (FIG. 6M).

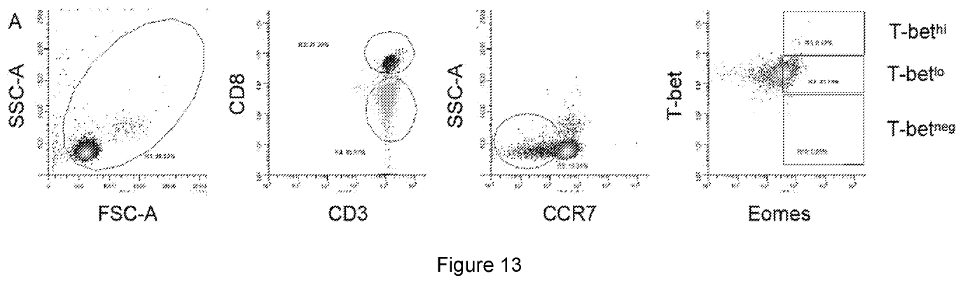

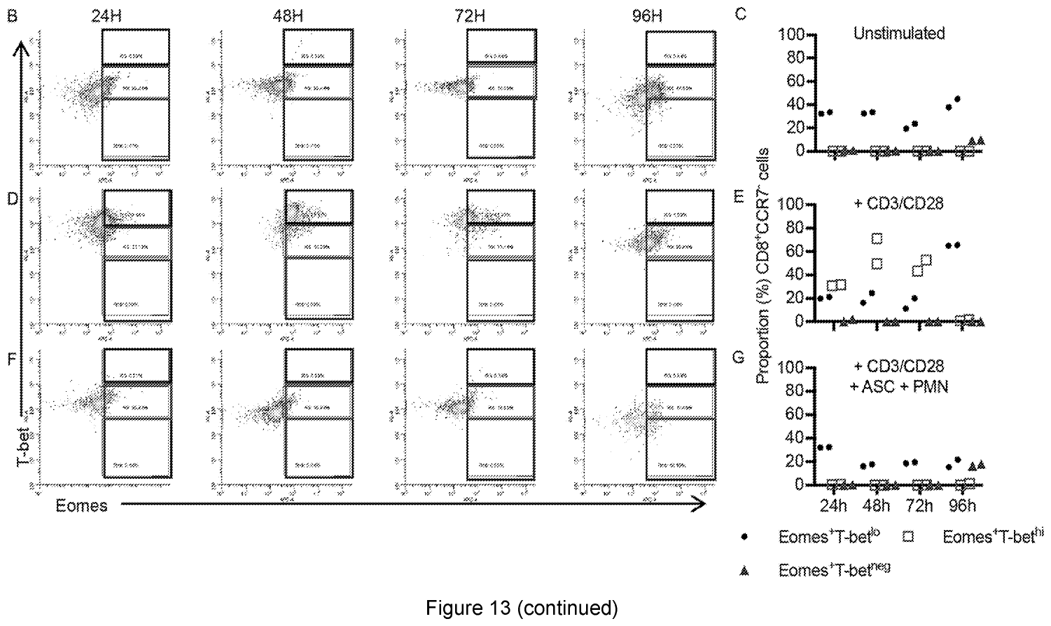

Next, we evaluated the transcriptional control of CD8⁺ effector differentiation, as measured by the upregulation of T-bet and Eomes (41). T-bet expression is associated with CTL differentiation while Eomes expression is associated with memory T cell differentiation. By gating on CD3⁺CD8⁺CCR7^{neg} T cells (FIG. 13A), anti-CD3/CD28-stimulation increased the proportion of Eomes⁺T-bet^{h} T cells by 24 h, as compared to unstimulated, and this increase contracted by 96 h (FIG. 13B-E). T cells cocultured with ascites and PMN phenocopied the Eomes⁺T-bet^{lo} signature of unstimulated cells (FIG. 13F-G), indicating that the PMN suppressor phenotype inhibits differentiation into CD8⁺ effector T cells. Ascites and/or PMN reduced the proportion of anti-CD3/CD28-stimulated CD8⁺ T cells expressing IFN-gamma (FIG. 6N). In addition, ascites or PMN alone both reduced anti-CD3/CD28-stimulated production of IL-2 by T cells at 24 and 72 h, while cocultures with ascites and PMN completely abrogated T cell IL-2 production (FIG. 60-P). Together, these results show that the PMN suppressor phenotype inhibits T cell activation independently of upregulation of exhaustion marker expression, and has broad inhibitory effects on T cell activation, including the suppression of effector differentiation and cytokine responses.

We evaluated whether the PMN suppressor phenotype affected tumor cell lysis. NY-ESO-1-specific CD8⁺ T cells from patients who received NY-ESO-1 vaccination were amplified in vitro and NYESO-1₁₅₇₋₁₆₅-specific CD8⁺ T cells were isolated as described (42). Using NYESO-1₁₅₇₋₁₆₅-specific CD8⁺ CTL and tumor cell (SK29) targets preloaded with NYESO-1157-165 peptide, we observed that PMN and/or ascites had no effect on antigen-specific cytotoxicity (FIG. 6Q). Together, these results show that the PMN suppressor phenotype suppressed the expansion and activation of T cells without affecting CTL activity.

### EXAMPLE 8

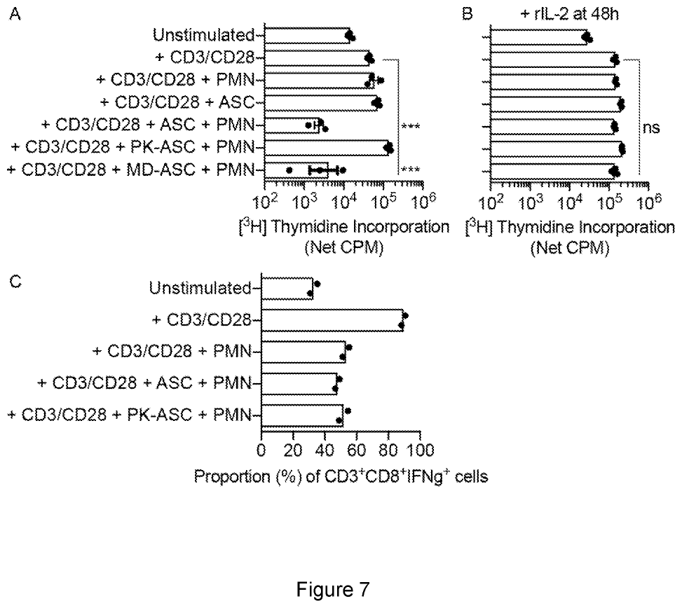

This example demonstrates that PMN suppressor phenotype inhibits the expansion of TCR-engineered CTL. To further understand how the PMN suppressor phenotype may be a barrier to immunotherapy, we evaluated the effect of cocultures with ascites and PMN on CTL with engineered TCR that recognize the tumor antigen, NY-ESO-1, and are in development for adoptive cellular therapy. Engineered CTL are activated during the expansion process prior to use in cocultures, accounting for the higher baseline proliferation observed in unstimulated cells and the modest increase in proliferation observed in anti-CD3/CD28-stimulated cells (FIG. 7A). The PMN suppressor phenotype inhibited stimulated proliferation of CTL below unstimulated levels, while neither ascites nor PMN alone had an effect on proliferation. PK-ASC abrogated the PMN suppressor phenotype, while MD-ASC had no effect, consistent with data in primary T cells. In contrast to cocultures with primary T cells where rIL-2 did not reverse T cell suppression, addition of rIL-2 to cocultures with engineered CTL at 48 h completely restored proliferation, suggesting that mechanisms for reversal of the PMN suppressor phenotype depends on activation status of the T cells (FIG. 7B). IFN-gamma expression was reduced to a similar level after cocultures with PMN and/or ascites (FIG. 7C). These results point to the PMN suppressor phenotype within the TME as a potential barrier to adoptive cellular therapy.

### EXAMPLE 9

This example demonstrates that post-operative drainage fluid induces the PMN suppressor phenotype.

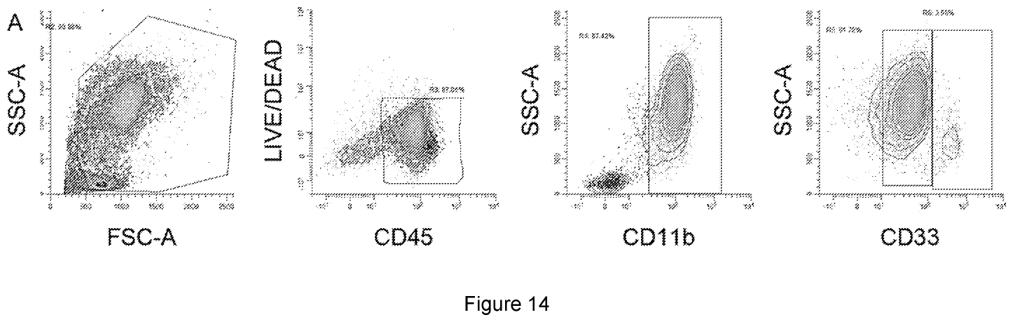

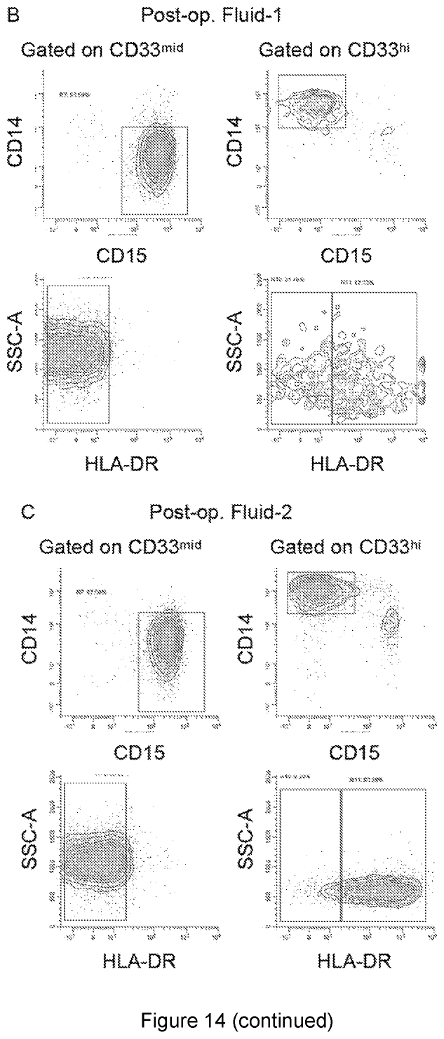

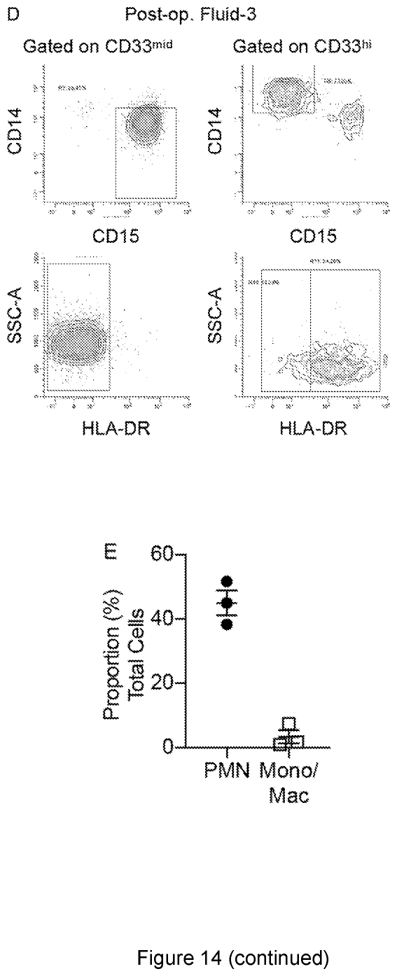

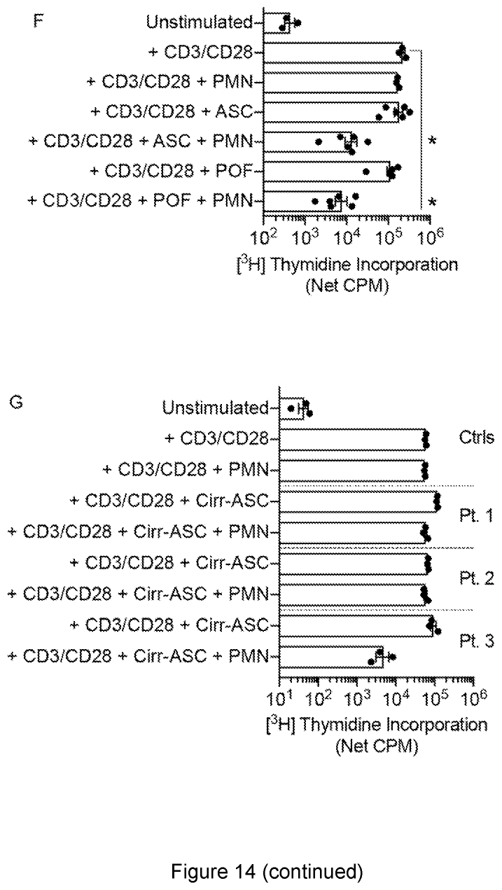

We analyzed whether the PMN suppressor phenotype was specific to the TME or instead a more general response to injury. We evaluated whether post-operative peritoneal fluid collected from a surgical drain 1d after primary surgery for EOC would induce the PMN suppressor phenotype. In contrast to ascites collected prior to surgery, which contained a mixed WBC population, post-operative drainage fluid indicated a neutrophilic peritonitis (FIG. 14A-E). The numbers of cells in the post-operative drainage fluid were insufficient for suppression studies. Therefore, we compared the capacity of paired ascites supernatants and post-operative drainage supernatants to induce the PMN suppressor phenotype. The debulking statuses of these patients were R0 (3/7), defined as no macroscopic residual tumor, or optimal (4/7), defined by remaining disease 0.1-1 cm. Similar to ascites, post-operative drainage supernatants were not suppressive alone, but induced PMN to suppress anti-CD3/CD28-stimulated T cell proliferation (FIG. 14F). To further probe whether the PMN suppressor phenotype was specific to the TME, we tested whether ascites supernatants from patients with cirrhosis and without cancer had the ability to induce the PMN suppressor phenotype. We observed T cell suppression in 1/3 samples tested (FIG. 14G). These findings support that inflammation and injury, whether resulting from the TME or other pathologic conditions, can induce the PMN suppressor phenotype.

### EXAMPLE 10

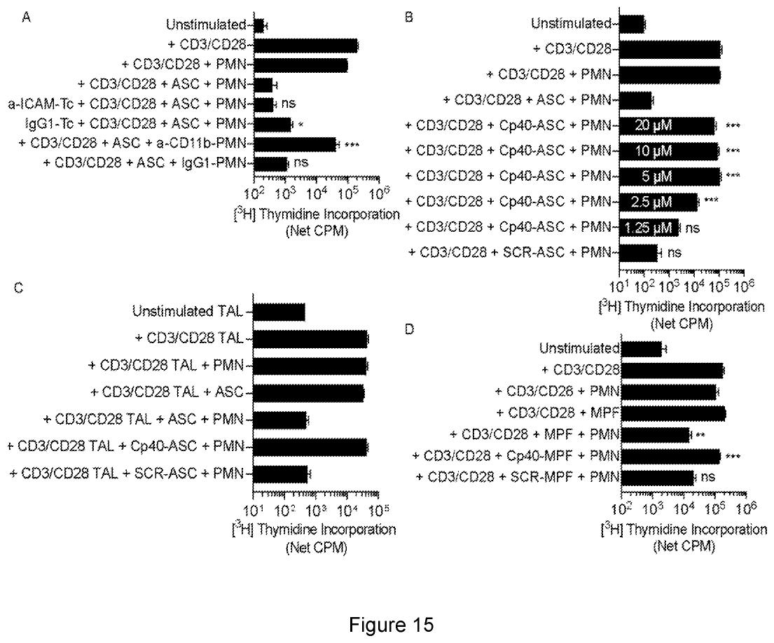

This Example demonstrates that inhibition of complement C3 activation abrogates the neutrophil suppressor phenotype induced by malignant effusions in patients with a number of metastatic cancers. Neutrophil-T cell contact was required for suppression. In addition to complement signaling, CR3 (CD11b/CD18 heterodimer) mediates a critical step in neutrophil recruitment and cell-cell adhesion by binding to ICAM-1 on endothelial cells. In coculture studies, pretreating neutrophils with neutralizing anti-CD11b abrogated suppression, while pretreating T cells with neutralizing anti-ICAM-1 had no effect (FIG. 15A). CR3 is also activated by iC3b, a cleavage product of C3b. To test the role of C3 activation, we treated ascites with Cp40, a compstatin in clinical trial development, prior to coculture with neutrophils and T cells. Cp40 completely abrogated the neutrophil suppressor phenotype, while scramble peptide had no effect (FIG. 15B). Neutrophil viability after 24 h exposure to Cp40-treated ascites was similar to neutrophils incubated with untreated ascites or scramble peptide-treated ascites. To investigate complement activation downstream of C3 on the neutrophil suppressor phenotype, we evaluated targeting of C5 or C7. Inhibition of C5 with an Ab or peptide inhibitor had a partial abrogating effect on T cell suppression, while inhibiting C7, a required constituent of the membrane attack complex (MAC), had no effect.

Inhibition of C3 abrogates neutrophil suppressor phenotype on EOC TALs In patients with metastatic EOC, tumor antigen-specific CD8⁺ T cells have impaired effector function and increased co-expression of PD-1 and LAG-3 compared to circulating lymphocytes, and effector function was enhanced with blockade of PD-1 and LAG-3. We confirmed high expression of PD-1 and LAG-3 on TALs from the ascites of patients with newly diagnosed EOC (not shown). We next cocultured cryopreserved or fresh TALs from 3 patients with autologous ascites and/or neutrophils from healthy donors. Similar to circulating lymphocytes, neutrophil suppressors inhibited stimulated proliferation of TALs, and suppression was fully abrogated by Cp40 (FIG. 15C). These results support neutrophil suppressors in the TME as obstacles to activation of TALs required for durable anti-tumor immunity and for the potential of C3 inhibition to overcome this barrier.

We observed a similar neutrophil suppressor phenotype when neutrophils and stimulated T cells were cocultured with pleural fluid from patients with a number of metastatic cancers (e.g., lung, breast, pancreatic), which was also abrogated by Cp40 (FIG. 15D). These results underscore the generalizability regarding complement-dependent neutrophil suppressor function in the TME.

### EXAMPLE 11

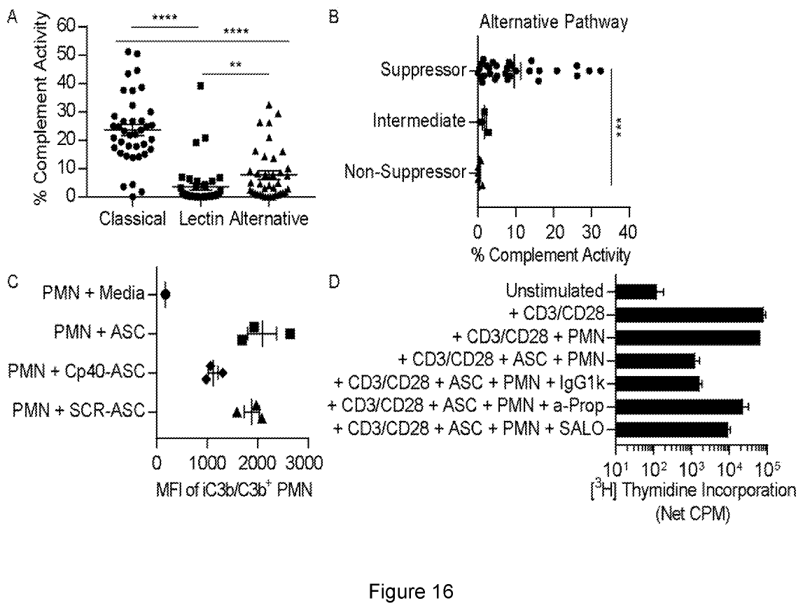

This example demonstrates that neutrophil suppressor phenotype is dependent on activation of alternative and classical complement pathways in ascites. EOC ascites have elevated levels of C3a and soluble C5b-9. We found that activation of complement in ascites principally occurs via the classical and alternative pathways (AP) (FIG. 16A). Activation of the AP was significantly higher in suppressor ascites vs. non-suppressor ascites, while classical pathway activity wasn't correlated with suppression (FIG. 16B). Ascites resulted in increased C3b/iC3b deposition on neutrophils that was reduced by Cp40 (FIG. 16C). We next evaluated upstream activators of C3 in the induction of neutrophil suppressor function. Properdin is stored in neutrophil secondary granules, and is required for activation of the AP C3 convertase. In neutrophil-T cell cocultures, anti-properdin resulted in a 1-log₁₀ increase in stimulated T cell proliferation vs. isotype. SALO, a peptide inhibitor of the classical pathway, had an intermediate effect, while peptide inhibitors of MASP-1 and MASP-2, required for lectin pathway activation, had no effect (not shown).

### EXAMPLE 12

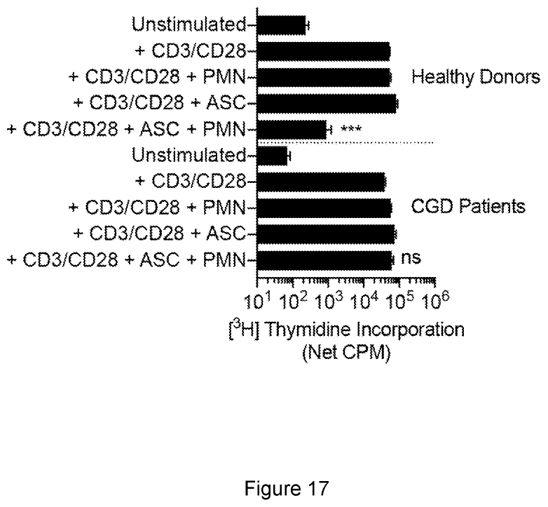

This example demonstrates that neutrophil NADPH oxidase is required for the neutrophil suppressor phenotype. Release of properdin from neutrophil secondary granules activates the AP and amplifies neutrophil activation, including NADPH oxidase activation. Chronic granulomatous disease (CGD) is an inherited disorder of the phagocyte NADPH oxidase. CGD neutrophils were unable to suppress autologous stimulated T cells after coculture with ascites as compared to neutrophils from the matched healthy donors (FIG. 17). These results support complement signaling mediating neutrophil suppressor function through NADPH oxidase.

### EXAMPLE 13

The following materials and methods were used to generate results described herein.

Patients and Specimens. Participants included healthy donors, control female patients with a benign adnexal mass undergoing resection surgery, and cancer patients with malignant effusions. Healthy donors (n=4) were Caucasian, aged 26-51, and equally divided between sexes. From 2015-2017, blood and ascites were collected from patients with newly diagnosed advanced (stage III or IV) ovarian cancer, as previously described (79). Blood was collected prior to primary surgery, and ascites were collected either by diagnostic paracentesis or in the operating room prior to surgery. Ascites were filtered through 300 μM filters and then centrifuged (500g, 10 min). Aliquots of supernatants were stored at −80° C. until further use. When available, post-operative drainage fluid from an abdominal drainage tube was collected the day after primary surgery. Patients with early stage (I or II) or unstaged disease were excluded from the analysis. The medical records of these patients were retrospectively reviewed for demographics, tumor stage and grade, baseline serum CA125 levels, debulking status, and chemotherapy response. In 2018, malignant pleural effusions were collected by thoracentesis from patients with various metastatic cancers and processed following the same protocol.

Analysis of Immune Infiltrate in Peripheral Blood and Ascites. Peripheral blood was collected in EDTA-coated tubes (Vacutainer, BD Biosciences, San Jose, Calif.). Whole blood was washed with PBS and centrifuged (500g, 10 min). Cells from blood and ascites were analyzed by flow cytometry within 24 h. Flow cytometry analysis was conducted on a Fortessa (Becton Dickinson, Franklin Lakes, N.J.). Forward scatter versus side scatter gating was set to include all non-aggregated cells from at least 20,000 events collected per sample. Data were analyzed using WinList 9.0.

Isolation of PMN and T Cells from Peripheral Blood

PMN and T cells were isolated from peripheral blood <1 h post-collection using the MACSxpress Neutrophil Isolation Kit and the CD4, CD8, or Pan T cell Isolation Kits, respectively (Miltenyi Biotec, Inc, Auburn, Calif., USA). The purity of PMN was >96% based on cytology and CD45⁺CD33^{mid}CD15⁺CD66b⁺(80); there was complete concordance between CD15 and CD66b expression. The purity of T cells was >97% based on CD45⁺CD3⁺, CD45⁺CD3⁺CD4⁺, and CD45⁺CD3⁺CD8⁺ expression.

Statistics

All statistical analyses were performed using the R 3.4.0 statistical computing language. A nominal significance threshold of 0.05 was used unless otherwise specified. Statistical testing utilized ANOVA to determine significance followed by a Tukey multiple comparisons post-test to determine which groups were significant. Pre-specified interactions were tested within the ANOVA framework. The multivariate analysis comprised FIGO stage, categorized as early (I, II, or IIIA/B) or late (IIIC or IV), histological grade, serum CA125 levels, debulking status (R0, defined as no macroscopic residual disease; optimal, defined by remaining disease 0.1-1 cm; and suboptimal, defined by remaining visible disease >1 cm), and platinum-sensitive versus refractory disease.

Study Approval

This study was approved by the Institutional Review Board (IRB) of Roswell Park Comprehensive Cancer Center (Roswell Park), Buffalo, NY, and was in compliance with federal and state requirements. All participants gave informed consent prior to inclusion in the study (protocols i215512 and i188310). All studies were conducted in compliance with the Declaration of Helsinki.

### EXAMPLE 14

This Example provides results that supplement the foregoing examples.

Cytology Slide Preparation, Staining, and Review

An Advia 120 or Advia 2120 Hematology System (Siemens) provided an automated WBC count on ascites from patients with newly diagnosed metastatic high-grade serous ovarian cancer (HGSOC), the most common histologic subtype of EOC. Cytospins were prepared using Cytopro slides and a Cytopro 760 Cytospin Centrifuge (Wescor). Slides were manually stained with Wright-Giemsa for morphologic evaluation. A hematopathologist (JTW) performed morphologic analysis of the slides. Differential counts (300 total cell counted) of the inflammatory cells in the ascites were performed. Tumor cells and mesothelial cells were excluded from the differential count.

Antibodies and Staining of Peripheral Blood and Ascites

Cells were subjected to RBC lysis, followed by washing and staining in buffer (DPBS, 1% BSA, 2 mM EDTA). Fc receptors were blocked to prevent non-specific antibody binding prior to staining (15 min, 4° C.; anti-mouse CD16/CD32, clone 2.4G2; BD Biosciences). Antibodies targeted to human CD45 (clone HI30), CD33 (P67.6), CD11b (CBRM1/5), CD15 (W6D3), CD14 (M5E2), and HLA-DR (L243) (Biolegend, San Diego, Calif.) were used to evaluate the proportion of granulocyte and monocyte/macrophage populations.

Coculture of PMN with Ascites and T Cells

Freshly isolated T cells (1e5) were stimulated with anti-CD3/CD28 Dynabeads (2.5 μl; Thermo Fisher Scientific) and cocultured with autologous PMN (1:1) and ascites supernatants (50% final well volume) in an incubator at 37° C., 5% CO₂. After 72 h, [³H] thymidine (1 μCi per well) was added and allowed to incorporate for 16-18 h. Cells were harvested onto a Filtermat and counted on a Beta counter. Results are expressed as net cpm [calculated by subtracting the average cpm of unstimulated T cells from the average cpm of stimulated T cells].

Where indicated, cells were pelleted for immunophenotyping and supernatants were saved for ELISA at time points throughout coculture. Viability was assessed by staining with Annexin V and/or PI (Dead Cell Apoptosis Kit, V13241, Thermo Fisher Scientific), following the manufacturer's protocols. For surface phenotyping and intracellular staining, cells were stained in buffer (DPBS, 1% BSA, 2 mM EDTA) and Cytofix/Cytoperm buffer kit (BD Biosciences), respectively, following the manufacturer's protocols. Data were analyzed using FCS Express 6.

Pretreatments of T cells, PMN, and Ascites Supernatants

Where indicated, T cells, PMN, and ascites supernatants underwent various pretreatments. Where indicated, T cells were pretreated for lh with neutralizing antibody against ICAM-I (1-10 μg), IgG1 isotype (1-10 μg), or media (RPMI 1640, 5-10% heat-inactivated FBS, HEPES, sodium pyruvate, non-essential amino acids, and penicillin-streptomycin).

Where indicated, PMN were pretreated for lh with neutralizing antibody against CD11b (1-10 μg), TGF-beta receptor 1 (1-10 μg) or IgG1 isotype (1-10 μg); thapsigargin (THG, 0.5-2 μM), diphenyleneiodonium (DPI, 1-25 μM), N-Formylmethionine-leucyl-phenylalanine (fMLF, 1-100 nM), brefeldin-A (1-10 μg/mL), ER export inhibitor 1 (Exol, 20-75 μM), CI-amidine (10-20 μM), SNARE decoys: TAT-SNAP23 (0.6-0.9 μg/mL), TAT-SYN4 (0.6-0.9 μg/mL), TAT-GST (0.6-0.9 μg/mL) (produced as described in the laboratory of Dr. Kenneth McLeish), puromycin and actinomycin-D (1-5 μg/mL), or with media.

Where indicated, ascites supernatants were pretreated with heat (56° C., lh) to denature heat-labile constituents or proteinase-K (100 μM, 37° C., 12 h) to degrade proteins. Two formulations of compstatin, a peptide C3 inhibitor, were used: CS (250 μM, 30° C., 2 h, Tocris) and Cp40 (1.25-20 μM, 30° C., 2 h), as well as a scramble peptide. In separate studies, ascites supernatants were ultra-centrifuged (100,000g, 4° C., 1.5 h) to separate the membrane-rich (MR-ASC) and membrane-depleted (MD-ASC) fractions. In others, ascites supernatants were pretreated for lh with neutralizing antibody against C5 (0.5-1.0 μg, A217; Quidel, San Diego, Calif.) or C7 (0.5-1.0 μg, A221; Quidel) or IgG1 isotype (0.5-1.0 μg), or with OmCI (0.6-1.2 μM), a peptide C5 inhibitor derived from the saliva of Ornithodoros moubata (35, 36).

Where indicated, L-arginine (50 μM, 1 mM), N-acetylcysteine (10-25 mM), DNase I (50-100 IU), rIL-2 (100 IU), Zileuton (50 μM), Indomethacin (10 μM), or 1-methyl-DL-tryptophan (1-MT; 100 μM) was added to the cocultures.

Antibodies and Staining of T Cells After Coculture

Fc receptors were blocked to prevent non-specific antibody binding prior to staining (15 min, 4° C.; anti-mouse CD16/CD32, clone 2.4G2; BD Biosciences). For phenotyping of PMN, antibodies targeted to human CD45 (clone REA747), CD15 (VIMC6) (Miltenyi Biotec, Inc.), CD63 (H5C6), CD66b (G10F5), and CD35 (Ell) (Biolegend) were used. For phenotyping of T cells, antibodies targeted to human CD45 (clone REA747), CD3 (REA613), CD4 (REA623), CD8 (REA734), CD62L (145/15), CD69 (REA824), CD4OL/CD154 (REA238), CD107a (REA792), PD-1/CD279 (PD1.3.1.3), LAG-3/CD223 (REA351), CTLA-4/CD152 (REA1003) (Miltenyi Biotec, Inc.), IFN-gamma (4S.B3) (Biolegend), T-bet (4B10), Eomes (WD1928), and CCR7/CD197 (4B12) (Thermo Fisher Scientific) were used.

Sorting of T cell Populations

Donor T cells (CD3⁺) isolated form peripheral blood were sorted (FACScan, Becton Dickinson) to isolate naive (CD3⁺CD45RA⁺RO⁻CD62L⁺), central memory (CD3⁺CD45RA⁻RO⁺CD62L⁺), and effector memory (CD3⁺CD45RA⁻RO⁺CD62L⁻) populations. Post-sort analysis using antibodies targeted to human CD3 (clone REA613), CD45RA (REA562), CD45RO (REA611), CD62L (145/15) (Miltenyi Biotec, Inc) showed >90% purity. Forward scatter versus side scatter gating was set to include all non-aggregated cells.

Measurement of Interleukin-2 by ELISA

Interleukin-2 (IL-2) levels from banked coculture supernatants were measured by Quantikine ELISA for Human IL-2, according to manufacturer's protocol (D2050, R&D Systems).

Preparation of PMNfor Proteomics Analysis

PMN were isolated from peripheral blood, as previously described. Ascites supernatants were pretreated with proteinase-K (100 μM, 37° C., 12 h). PMN (5e6; 5 technical replicates) were exposed to untreated ascites supernatants, proteinase-K-digested ascites supernatants, or media (RPMI 1640, 5-10% heat-inactivated FBS, HEPES, sodium pyruvate, non-essential amino acids, and penicillin-streptomycin) for 30 or 60 min. PMN were washed, decanted of all liquid, flash frozen on dry ice, and stored at −80° C. for subsequent proteomic analysis.

PMN (5e6) were resuspended in 1 mL surfactant cocktail buffer containing 50 mM Tris-formic acid (FA; pH 8.0) containing 150 mM NaCl, 2% sodium dodecyl sulfate (SDS), 0.5% SDC, and 2% IGEPAL CA-630, with complete protease inhibitor cocktail (Roche Applied Science, Indianapolis, Ind.). Samples were placed on ice for 10 min, and then sonicated with 5 sonication-chill cycles until the liquid became pellucid. The sonicated samples were placed on ice for another 30 min, and then centrifuged (20,000g, 30 min, 4° C.). Supernatants were carefully transferred to new low-bind tubes, and the protein concentration of all samples was determined by bicinchoninic acid assay (BCA; Pierce Biotechnology, Inc., Rockford, Ill.).

For each sample, 100 μg of extracted proteins (normalized to 1 μg/μL by 0.5% SDS) were used for LC-MS analysis. Proteins were first reduced with 10 mM dithiothreitol (DTT; 30 min, 56° C.) and alkylated using 20 mM iodoacetamide (IAM; 30 min, 37° C.). Both steps were performed while covered with aluminum foil with constant agitation. Denatured proteins were precipitated by the addition of 7 volumes of chilled acetone, followed by incubation (3 h, −20° C.). After centrifugation (20,000 g, 30 min, 4° C.), the pelleted proteins were washed with 500 μL methanol, briefly air-dried, and resuspended in 80 μL 50 mM Tris-FA. A total of 5 μg trypsin (Sigma-Aldrich, St. Louis, Mo.) dissolved in 20 μL 50 mM Tris-FA was added to the protein pellets at a final enzyme: substrate (E:S) ratio of 1:20, and the proteins were incubated (18 h, 37° C.) with constant vortexing. Derived peptides were acidified by adding 1% FA, centrifuged (20,000 g, 30 min, 4° C.) and transferred to LC vials.

Proteomics LC-MS Analysis

RPLC separation of derived peptides was performed on a Dionex Ultimate 3000 nano LC system and an Ultimate 3000 gradient micro LC system with a WPS-3000 autosampler (Thermo Fisher Scientific, San Jose, Calif.). Mobile phase A and B were 0.1% formic acid in 2% acetonitrile and 0.1% FA in 88% acetonitrile. A total of 4 μg peptides were loaded onto an RP trap (300 μm ID×1 cm), with 1% mobile phase B at a flow rate of 10 μL/min, and the trap was washed for 3 min. A series of nano-flow gradients (flow rate at 250 nL/min) was set to back-flush the trapped samples onto the nano-LC column (75-μm ID×100 cm), which was heated at 52° C. to improve chromatographic resolution and reproducibility. The optimized gradient profile was 4-13% B for 15 min; 13-28% B for 110 min; 28-44% B for 5 min; 44-60% B for 5 min; 60-97% B for 1 min, and isocratic at 97% B for 17 min. Peptides eluted from nano-LC was analyzed by an Orbitrap Fusion Lumos mass spectrometer (Thermo Fisher Scientific, San Jose, Calif.). MS was operated under the data dependent mode. MS1 spectra were collected at 120,000 resolution with an automated gain control (AGC) target of 500,000 and a max injection time of 50 ms. Previously interrogated precursors were excluded using a dynamic window (60s±10 ppm). For MS2, precursor isolation window was set to 1 Th, and precursor ions were fragmented by high-energy collision dissociation (HCD) at a normalized collision energy of 35%. MS2 spectra were collected at 15,000 resolution with an AGC target of 50,000 and a max injection time of 50 ms.

LC-MS raw files were processed with an in-house developed MS1-based label-free quantification pipeline, IonStar. Peptide identification was performed by MS-GF+ (v9979, released on 03/26/2014) searching against a Uniprot-Swissprot Homo sapiens protein database (20200 protein entries) concatenated with reversed decoy sequences for false discovery rate (FDR) control. Parameters follow the default setting of MS-GF+ except the following ones: 20 ppm for precursor mass tolerance, −1 to 2 for isotope error range, fully-tryptic peptides only, 2 to 7 for precursor charge state, cysteine carbamidomethylation for fixed modification, methionine oxidation and peptide N-terminal acetylation for variable modification. Peptide filtering, protein grouping and protein-level FDR control were conducted by IDPicker (v3.1.643.0). Minimal peptide number for identification was set to 2, and global protein-level FDR was set to 1%. Filtered PSM/peptide/protein lists were exported and combined into a spectrum report using an in-house R script.

For quantification, rawfiles were imported into SIEVETM (v2.2, Thermo Scientific) for global chromatographic alignment by ChromAlign and quantitative feature generation by a direct ion-current extraction (DICE) method. To ensure reliable quantification, only features with high quality (e.g. S/N ratio >10) were generated, and ion intensities in each sample run was calculated individually. A customized R package,

IonStarStat, was used to integrate quantitative features with identification results, perform dataset-wide normalization, remove outlier peptides from quantification, and aggregate peptide intensities to protein level. Protein ratios and p-values (from paired t-test) between ascites/PK ascites and media control were calculated manually in Excel. Significantly changed proteins were determined by protein fold change >1.5 and p-value <0.05. Gene ontology (GO) analysis was performed by the Database for Annotation, Visualization and Integrated Discovery (DAVID) Bioinformatics Resources v6.7 (http://david.abcc.ncifcrf.gov).

Cytotoxicity Assay

The in vitro cytotoxicity assay was performed using the CFSE-based assay, as previously described (43). Briefly, NY-ESO-1-specific CD8⁺ T cells from patients who received NY-ESO-1 vaccination were amplified in vitro and NYESO-1₁₅₇₋₁₆₅-specific CD8⁺ T cells were isolated. HLA-A*0201⁺NYE-ESO-1⁻SK-MEL-29 (SK29) cells were pulsed with NY-ESO-1₁₅₇₋₁₆₅ peptide for 2 h in an incubator at 37° C., 5% CO₂ followed by labeling with 0.5 μM CF SE. Peptide-unpulsed SK29 cells were labeled with 5 μM CF SE. Peptide-pulsed (2e4) and unpulsed (1:1) SK29 cells were cocultured with NY-ESO-1₁₅₇₋₁₆₅-specific CD8⁺ T cells (4e4) in the presence or absence of ascites supernatants (50 μl) and/or PMN (1:1) for 16-18 h. The cells were harvested by treatment with trypsin/EDTA, resuspended in buffer (DPBS, 1% heat-inactivated FBS), and stained with 7-AAD (BD Biosciences). The cells were acquired on a FACSCalibur flow cytometer (BD Biosciences) and the proportion of CFSE⁺7-AAD⁻ cells were analyzed by FlowJo software. Cytotoxicity was calculated using the following formula: % cytotoxicity=100x[1-(%CFSE^{hi} peptide-unpulsed SK29/% CFSE^{lo} peptide-pulsed SK29) without T cells/(% CFSE^{hi} peptide-unpulsed SK29/%CFSE^{lo} peptide-pulsed SK29)with T cells].

Engineered T Cells with NY-ESO-1-Specific TCR

Peripheral blood mononuclear cells (PBMC) from healthy donors were obtained through Ficoll separation. PBMC were activated with OKT3 (anti-CD3 Ab, 50 ng/mL) and rIL-2 (300 IU/mL) for 48 h in an incubator at 37° C., 5% CO₂. Supernatants from the retrovirus construct MSCV-NY-ESO-1 TCR (collected from a stable PG13 producer cell line) were added to retronectin precoated plates, and the PBMC were introduced suspended in AIMV media (5% AB serum). The cells were centrifuged for spinoculation and kept for 16 h in an incubator at 37° C., 5% CO₂, then washed and resuspended in fresh AIMV media (5% AB serum) for downstream applications.

TABLE 1
Patients with newly diagnosed EOC have normal circulating WBC
numbers and differentials. Data are based on review of electronic
health records at Roswell Park Comprehensive Cancer Center. Statistical
comparisons were by ANOVA with Tukey post-test.

| | Suppressor Status | | | |
|---|---|---|---|---|
| | Suppressor | Intermediate | Non | |
| | (x ≥ 1 | (0.5 log₁₀ ≤ | (x < 0.5 | p- |
| | log₁₀) | x < 1 log₁₀) | log₁₀) | value |
| N (31) | 20 | 8 | 3 | |
| Pre-operative CBC | 16/20 | 7/8 | 3/3 | |
| WBC | 8.6 | 8.5 | 9.3 | 0.94 |
| (×10\hat{ }9 cells/L) | | | | |
| % HCT | 36.1 | 35.6 | 39.6 | 0.62 |
| PLT | 411.0 | 565.0 | 352.3 | 0.22 |
| (×10\hat{ }9 cells/L) | | | | |
| Differential | 13/20 | 6/8 | 3/3 | |
| PMN | 6.3 | 6.5 | 6.1 | 0.93 |
| (×10\hat{ }9 cells/L) | | | | |
| Lymphocyte | 1.3 | 1.2 | 2.2 | 0.56 |
| (×10\hat{ }9 cells/L) | | | | |
| Monocyte | 0.6 | 0.5 | 0.7 | 0.71 |
| (×10\hat{ }9 cells/L) | | | | |
| NLR | 4.7 | 5.5 | 2.8 | 0.73 |
| Manual Counts | 7/20 | 3/8 | 1/3 | |
| % Segmented | 67.0 | 77.5 | 73.2 | 0.30 |
| PMN | | | | |
| % Bands | 1.0 | NR | NR | |
| % | NR | NR | NR | |
| Metamyelocytes | | | | |

CBC, complete blood cell count; WBC, white blood cell; HCT, hematocrit; PLT, platelet; NLR, neutrophil-to-lymphocyte ratio; NR, none recorded.

TABLE 2
Ascites stratification strategy based on the
induction of a suppressor phenotype in PMN.

| | Suppressor Status | | | |
|---|---|---|---|---|
| | Suppressor | Intermediate | Non | |
| | (x ≥ 1 | (0.5 log₁₀ ≤ | (x < 0.5 | p- |
| | log₁₀) | x < 1 log₁₀) | log₁₀) | value |
| N (31) | 20 | 8 | 3 | |
| Age, Mean | 65.4 | 69.6 | 64.0 | 0.39 |
| Histology | | | | 0.40 |
| EOC, serous | 15 | 6 | 2 | |
| EOC, non-serous | 2 | 0 | 0 | |
| OC, non-epithelial | 3 | 2 | 0 | |
| Benign, thecoma | 0 | 0 | 1 | |
| Stage | | | | 0.62 |
| IIIA | 1 | 0 | 0 | |
| IIIB | 1 | 1 | 0 | |
| IIIC | 16 | 6 | 2 | |
| IV | 2 | 1 | 0 | |
| Grade 3 | 19 | 8 | 2 | 1.00 |
| CA125 | 576.7 (n = | 360.5 (n = | 9251.1 (n = | 0.44 |
| | 12) | 5) | 1) | |
| Debulking Surgery | 15/20 | 6/8 | 1/3 | |
| % R0 | 46.7 | 16.7 | 0 | 0.14 |
| % Optimal | 40.0 | 50.0 | 100.0 | 0.57 |
| % Suboptimal | 13.3 | 33.3 | 0 | 0.44 |
| Chemotherapy | 12/20 | 2/8 | 1/3 | |
| Response* | | | | |
| % Platinum | 75.0 | 100.0 | 100.0 | 0.58 |
| Sensitive | | | | |
| % Platinum | 16.7 | 0 | 0 | 0.44 |
| Resistant | | | | |
| % Platinum | 8.3 | 0 | 0 | 0.60 |
| Refractory | | | | |

Ascites were stratified into three categories based on the induction of a suppressor phenotype in PMN, where x equals a reduction in proliferation as compared to anti-CD3/CD28-stimulated T cells alone: suppressors (x ≥ 1 log₁₀), intermediate suppressors (0.5 log₁₀ ≤ x < 1 log₁₀), and non-suppressors (x < 0.5 log₁₀). Statistical comparisons were by ANOVA with Tukey post-test.
*RECiST Evaluation complete response after end of adjuvant chemotherapy

TABLE 3
Clinical characteristics of patients with malignant
effusions that induced the PMN suppressor phenotype in
a complement C3-dependent mechanism.
Demographics of Patients with Malignant
Effusions other than EOC Ascites

| | |
|---|---|
| N | 7 |
| Age, Mean (Y) | 68.4 |
| Sex (Female:Male) | 5:2 |
| Type of Malignant Effusions | |
| Peritoneal Ascites | 1 |
| Pleural Effusion | 6 |
| Metastatic Cancer | |
| Ovarian | 2 |
| Lung | 2 |
| Pancreatic* | 1 |
| GI | 1 |
| Lymphoma | 1 |
| Newly Diagnosed | 3 |
| Received Prior Treatment | 4 |
| (e.g., surgery, chemotherapy, | |
| immunotherapy) | |

*Ascites

The following references are cited in this disclosure. This reference listing is not an indication that any of the references are material to patentability.
1. Jimenez-Sanchez A, Memon D, Pourpe S, Veeraraghavan H, Li Y, Vargas H A, Gill M B, Park K J, Zivanovic O, Konner J, et al. Heterogeneous Tumor-Immune Microenvironments among Differentially Growing Metastases in an Ovarian Cancer Patient. Cell. 2017;170(5):927-38.e20.
2. Worzfeld T, Pogge von Strandmann E, Huber M, Adhikary T, Wagner U, Reinartz S, and Muller R. The Unique Molecular and Cellular Microenvironment of Ovarian Cancer. Front Oncol. 2017;7(
3. Idorn M, Olsen M, Halldorsdottir H R, Skadborg S K, Pedersen M, Hogdall C, Hogdall E, Met O, and Thor Straten P. Improved migration of tumor ascites lymphocytes to ovarian cancer microenvironment by CXCR2 transduction. Oncoimmunology. 2018;7(4):e1412029.
4. Obermajer N, Muthuswamy R, Odunsi K O, Edwards R, and Kalinski P. PGE2-Dependent CXCL12 Production and CXCR4 Expression Control the Accumulation of Human MDSCs in Ovarian Cancer Environment. Cancer research. 2011.
5. Khan A N, Kolomeyevskaya N, Singel K L, Grimm M J, Moysich K B, Daudi S, Grzankowski K S, Lele S, Ylagan L, Webster G A, et al. Targeting myeloid cells in the tumor microenvironment enhances vaccine efficacy in murine epithelial ovarian cancer. Oncotarget. 2015.
6. Shenoy G N, Loyall J, Maguire O, Iyer V, Kelleher R J, Jr., Minderman H, Wallace P K, Odunsi K, Balu-Iyer S V, and Bankert R B. Exosomes Associated with Human Ovarian Tumors Harbor a Reversible Checkpoint of T-cell Responses. Cancer Immunol Res. 2018;6(2):236-47.
7. Shender V O, Pavlyukov M S, Ziganshin R H, Arapidi G P, Kovalchuk S I, Anikanov N A, Altukhov I A, Alexeev D G, Butenko I O, Shavarda A L, et al. Proteome-metabolome profiling of ovarian cancer ascites reveals novel components involved in intercellular communication. Mol Cell Proteomics. 2014;13(12):3558-71.
8. Biskup K, Braicu E I, Sehouli J, Tauber R, and Blanchard V. The ascites N-glycome of epithelial ovarian cancer patients. Journal of proteomics. 2017;157(33-9.
9. Worzfeld T, Finkernagel F, Reinartz S, Konzer A, Adhikary T, Nist A, Stiewe T, Wagner U, Looso M, Graumann J, et al. Proteotranscriptomics Reveal Signaling Networks in the Ovarian Cancer Microenvironment. Mol Cell Proteomics. 2018;17(2):270-89.
10. Huang H, Li Y J, Lan C Y, Huang Q D, Feng Y L, Huang Y W, and Liu J H. Clinical significance of ascites in epithelial ovarian cancer. Neoplasma. 2013;60(5):546-52.
11. Janco J M, Glaser G, Kim B, McGree M E, Weaver A L, Cliby W A, Dowdy S C, and Bakkum-Gamez J N. Development of a prediction model for residual disease in newly diagnosed advanced ovarian cancer. Gynecol Oncol. 2015;138(1):70-7.
12. Szender J B, Emmons T, Belliotti S, Dickson D, Khan A, Morrell K, Khan A, Singel K L, Mayor P C, Moysich K B, et al. Impact of ascites volume on clinical outcomes in ovarian cancer: A cohort study. Gynecol Oncol. 2017.
13. Zhang L, Conejo-Garcia J R, Katsaros D, Gimotty P A, Massobrio M, Regnani G, Makrigiannakis A, Gray H, Schlienger K, Liebman M N, et al. Intratumoral T cells, recurrence, and survival in epithelial ovarian cancer. The New England journal of medicine. 2003;348(3):203-13.
14. Sato E, Olson S H, Ahn J, Bundy B, Nishikawa H, Qian F, Jungbluth A A, Frosina D, Gnjatic S, Ambrosone C, et al. Intraepithelial CD8+ tumor-infiltrating lymphocytes and a high CD8+/regulatory T cell ratio are associated with favorable prognosis in ovarian cancer. Proceedings of the National Academy of Sciences of the United States of America. 2005;102(51):18538-43.
15. Curiel TJ, Coukos G, Zou L, Alvarez X, Cheng P, Mottram P, Evdemon-Hogan M, Conejo-Garcia J R, Zhang L, Burow M, et al. Specific recruitment of regulatory T cells in ovarian carcinoma fosters immune privilege and predicts reduced survival. Nature medicine. 2004;10(9):942-9.
16. Matsuzaki J, Gnjatic S, Mhawech-Fauceglia P, Beck A, Miller A, Tsuji T, Eppolito C, Qian F, Lele S, Shrikant P, et al. Tumor-infiltrating NY-ESO-1-specific CD8+ T cells are negatively regulated by LAG-3 and PD-1 in human ovarian cancer. Proceedings of the National Academy of Sciences of the United States of America. 2010;107(17):7875-80.
17. Brahmer J R, Tykodi SS, Chow L Q, Hwu W J, Topalian S L, Hwu P, Drake C G, Camacho L H, Kauh J, Odunsi K, et al. Safety and activity of anti-PD-L1 antibody in patients with advanced cancer. The New England journal of medicine. 2012;366(26):2455-65.
18. Hamanishi J, Mandai M, Ikeda T, Minami M, Kawaguchi A, Murayama T, Kanai M, Mori Y, Matsumoto S, Chikuma S, et al. Safety and Antitumor Activity of Anti-PD-1 Antibody, Nivolumab, in Patients With Platinum-Resistant Ovarian Cancer. J Clin Oncol. 2015;33(34):4015-22.
19. Veglia F, Perego M, and Gabrilovich D. Myeloid-derived suppressor cells coming of age. Nature immunology. 2018;19(2):108-19.
20. Bronte V, Brandau S, Chen S H, Colombo M P, Frey A B, Greten T F, Mandruzzato S, Murray P J, Ochoa A, Ostrand-Rosenberg S, et al. Recommendations for myeloid-derived suppressor cell nomenclature and characterization standards. Nat Commun. 2016;7(12150.
21. Wu W C, Sun HW , Chen H T, Liang J, Yu X J, Wu C, Wang Z, and Zheng L. Circulating hematopoietic stem and progenitor cells are myeloid-biased in cancer patients. Proceedings of the National Academy of Sciences of the United States of America. 2014;111(11):4221-6.
22. Netherby C S, Messmer M N, Burkard-Mandel L, Colligan S, Miller A, Cortes Gomez E, Wang J, Nemeth M J, and Abrams S I. The Granulocyte Progenitor Stage Is a Key Target of IRF8-Mediated Regulation of Myeloid-Derived Suppressor Cell Production. J Immunol. 2017;198(10):4129-39.
23. Cui TX, Kryczek I, Zhao L, Zhao E, Kuick R, Roh M H, Vatan L, Szeliga W, Mao Y, Thomas DG, et al. Myeloid-derived suppressor cells enhance stemness of cancer cells by inducing microRNA101 and suppressing the corepressor CtBP2. Immunity. 2013;39(3):611-21.
24. Kryczek I, Wei S, Zhu G, Myers L, Mottram P, Cheng P, Chen L, Coukos G, and Zou W. Relationship between B7-H4, regulatory T cells, and patient outcome in human ovarian carcinoma. Cancer research. 2007;67(18):8900-5.
25. Banerjee S, Rustin G, Paul J, Williams C, Pledge S, Gabra H, Skailes G, Lamont A, Hindley A, Goss G, et al. A multicenter, randomized trial of flat dosing versus intrapatient dose escalation of single-agent carboplatin as first-line chemotherapy for advanced ovarian cancer: an SGCTG (SCOTROC 4) and ANZGOG study on behalf of GCIG. Ann Oncol. 2013;24(3):679-87.
26. Huang Q T, Zhou L, Zeng W J, Ma Q Q, Wang W, Zhong M, and Yu Y H. Prognostic Significance of Neutrophil-to-Lymphocyte Ratio in Ovarian Cancer: A Systematic Review and Meta-Analysis of Observational Studies. Cellular physiology and biochemistry: international journal of experimental cellular physiology, biochemistry, and pharmacology. 2017;41(6):2411-8.
27. Shaul M E, Levy L, Sun J, Mishalian I, Singhal S, Kapoor V, Horng W, Fridlender G, Albelda S M, and Fridlender Z G. Tumor-associated neutrophils display a distinct N1 profile following TGFbeta modulation: A transcriptomics analysis of pro-vs. antitumor TANs. Oncoimmunology. 2016;5(11):e1232221.
28. Schmielau J, and Finn O J. Activated granulocytes and granulocyte-derived hydrogen peroxide are the underlying mechanism of suppression of t-cell function in advanced cancer patients. Cancer research. 2001; 61(12): 4756-60.
29. Rotondo R, Bertolotto M, Barisione G, Astigiano S, Mandruzzato S, Ottonello L, Dallegri F, Bronte V, Ferrini S, and Barbieri O. Exocytosis of azurophil and arginase 1-containing granules by activated polymorphonuclear neutrophils is required to inhibit T lymphocyte proliferation. J Leukoc Biol. 2011;89(5):721-7.
30. Tak T, Rygiel T P, Karnam G, Bastian O W, Boon L, Viveen M, Coenjaerts F E, Meyaard L, Koenderman L, and Pillay J. Neutrophil-mediated Suppression of Influenza-induced Pathology Requires CD11b/CD18 (MAC-1). American journal of respiratory cell and molecular biology. 2018;58(4):492-9.
31. Uriarte S M, Rane M J, Luerman G C, Barati M T, Ward R A, Nauseef W M, and McLeish K R. Granule exocytosis contributes to priming and activation of the human neutrophil respiratory burst. J lmmunol. 2011;187(1):391-400.
32. McLeish K R, Uriarte S M, Tandon S, Creed T M, Le J, and Ward R A. Exocytosis of neutrophil granule subsets and activation of prolyl isomerase 1 are required for respiratory burst priming. Journal of innate immunity. 2013;5(3):277-89.
33. Yang L, Zhang X, Ma Y, Zhao X, Li B, and Wang H. Ascites promotes cell migration through the repression of miR-125b in ovarian cancer. Oncotarget. 2017;8(31):51008-15.
34. McLane L M, Banerjee P P, Cosma G L, Makedonas G, Wherry E J, Orange J S, and Betts M R. Differential localization of T-bet and Eomes in CD8 T cell memory populations. J Immunol. 2013;190(7):3207-15.
35. Lizcano A, Secundino I, Dohrmann S, Corriden R, Rohena C, Diaz S, Ghosh P, Deng L, Nizet V, and Varki A. Erythrocyte sialoglycoproteins engage Siglec-9 on neutrophils to suppress activation. Blood. 2017;129(23):3100-10.
36. Negorev D, Beier U H, Zhang T, Quatromoni J G, Bhojnagarwala P, Albelda S M, Singhal S, Eruslanov E, Lohoff F W, Levine M H, et al. Human neutrophils can mimic myeloid-derived suppressor cells (PMN-MDSC) and suppress microbead or lectin-induced T cell proliferation through artefactual mechanisms. Sci Rep. 2018;8(1):3135.
37. Reis E S, Mastellos D C, Ricklin D, Mantovani A, and Lambris J D. Complement in cancer: untangling an intricate relationship. Nature reviews Immunology. 2018;18(1):5-18.
38. Yuen J, Pluthero F G, Douda D N, Riedl M, Cherry A, Ulanova M, Kahr W H, Palaniyar N, and Licht C. NETosing Neutrophils Activate Complement Both on Their Own NETs and Bacteria via Alternative and Non-alternative Pathways. Front Immunol. 2016;7(137.
39. Blatt A Z, Saggu G, Cortes C, Herbert A P, Kavanagh D, Ricklin D, Lambris J D, and Ferreira V P. Factor H C-Terminal Domains Are Critical for Regulation of Platelet/Granulocyte Aggregate Formation. Front Immunol. 2017;8(1586.
40. Cho M S, Vasquez H G, Rupaimoole R, Pradeep S, Wu S, Zand B, Han H D, Rodriguez-Aguayo C, Bottsford-Miller J, Huang J, et al. Autocrine effects of tumor-derived complement. Cell reports. 2014;6(6):1085-95.
41. Cho M S, Rupaimoole R, Choi H J, Noh K, Chen J, Hu Q, Sood AK, and Afshar-Kharghan V. Complement Component 3 Is Regulated by TWIST1 and Mediates Epithelial-Mesenchymal Transition. J Immunol. 2016;196(3):1412-8.
42. Markiewski M M, DeAngelis R A, Benencia F, Ricklin-Lichtsteiner S K, Koutoulaki A, Gerard C, Coukos G, and Lambris J D. Modulation of the antitumor immune response by complement. Nature immunology. 2008;9(11):1225-35.
43. Kwak J W, Laskowski J, Li H Y, McSharry M V, Sippel T R, Bullock B L, Johnson A M, Poczobutt J M, Neuwelt A J, Malkoski S P, et al. Complement Activation via a C3a Receptor Pathway Alters CD4(+) T Lymphocytes and Mediates Lung Cancer Progression. Cancer research. 2018;78(1):143-56.
44. Ajona D, Ortiz-Espinosa S, Moreno H, Lozano T, Paj ares M J, Agorreta J, Bertolo C, Lasarte J J, Vicent S, Hoehlig K, et al. A Combined PD-1/C5a Blockade Synergistically Protects against Lung Cancer Growth and Metastasis. Cancer discovery. 2017;7(7):694-703.
45. Liu CF, Min X Y, Wang N, Wang J X, Ma N, Dong X, Zhang B, Wu W, Li Z F, Zhou W, et al. Complement Receptor 3 Has Negative Impact on Tumor Surveillance through Suppression of Natural Killer Cell Function. Front Immunol. 2017;8(1602.
46. Platt J L, Silva I, Balin S J, Lefferts A R, Farkash E, Ross T M, Carroll M C, and Cascalho M. C3d regulates immune checkpoint blockade and enhances antitumor immunity. JCI insight. 2017;2(9).
47. Nunez-Cruz S, Gimotty P A, Guerra M W, Connolly D C, Wu Y Q, DeAngelis R A, Lambris J D, Coukos G, and Scholler N. Genetic and pharmacologic inhibition of complement impairs endothelial cell function and ablates ovarian cancer neovascularization. Neoplasia. 2012;14(11):994-1004.
48. Singel K L, and Segal B H. Neutrophils in the tumor microenvironment: trying to heal the wound that cannot heal. Immunological reviews. 2016;273(1):329-43.
49. Coffelt S B, Kersten K, Doornebal C W, Weiden J, Vrijland K, Hau C S, Verstegen N J, Ciampricotti M, Hawinkels L J, Jonkers J, et al. IL-17-producing gammadelta T cells and neutrophils conspire to promote breast cancer metastasis. Nature. 2015;522(7556):345-8.
50. Wculek S K, and Malanchi I. Neutrophils support lung colonization of metastasis-initiating breast cancer cells. Nature. 2015;528(7582):413-7.
51. Steele C W, Karim S A, Leach J D, Bailey P, Upstill-Goddard R, Rishi L, Foth M, Bryson S, McDaid K, Wilson Z, et al. CXCR2 Inhibition Profoundly Suppresses Metastases and Augments Immunotherapy in Pancreatic Ductal Adenocarcinoma. Cancer Cell. 2016;29(6):832-45.
52. Nywening T M, Belt B A, Cullinan D R, Panni R Z, Han B J, Sanford D E, Jacobs R C, Ye J, Patel A A, Gillanders W E, et al. Targeting both tumour-associated CXCR2(+) neutrophils and CCR2(+) macrophages disrupts myeloid recruitment and improves chemotherapeutic responses in pancreatic ductal adenocarcinoma. Gut. 2017.
53. Mcgray A R, Eppolito C, Miliotto A, Huang R, Singel K, Pol J, Stephenson K, Segal B H, Lichty B, and Odunsi K. AACR Annual Meeting. Washington, D.C.: American Association for Cancer Research; 2017.
54. Clark R A, and Klebanoff S J. Neutrophil-mediated tumor cell cytotoxicity: role of the peroxidase system. The Journal of experimental medicine. 1975;141(6):1442-7.
55. Eruslanov E B, Bhojnagarwala P S, Quatromoni J G, Stephen T L, Ranganathan A, Deshpande C, Akimova T, Vachani A, Litzky L, Hancock W W, et al. Tumor-associated neutrophils stimulate T cell responses in early-stage human lung cancer. The Journal of clinical investigation. 2014;124(12):5466-80.
56. Sagiv J Y, Michaeli J, Assi S, Mishalian I, Kisos H, Levy L, Damti P, Lumbroso D, Polyansky L, Sionov R V, et al. Phenotypic diversity and plasticity in circulating neutrophil subpopulations in cancer. Cell reports. 2015;10(4):562-73.
57. Kolomeyevskaya N, Eng K H, Khan A N, Grzankowski K S, Singel K L, Moysich K, and Segal B H. Cytokine profiling of ascites at primary surgery identifies an interaction of tumor necrosis factor-alpha and interleukin-6 in predicting reduced progression-free survival in epithelial ovarian cancer. Gynecol Oncol. 2015.
58. Michaeli J, Shaul M E, Mishalian I, Hovav A H, Levy L, Zolotriov L, Granot Z, and Fridlender Z G. Tumor-associated neutrophils induce apoptosis of non-activated CD8 T-cells in a TNFalpha and NO-dependent mechanism, promoting a tumor-supportive environment. Oncoimmunology. 2017;6(11):e1356965.
59. Lindemann S W, Yost C C, Denis M M, McIntyre T M, Weyrich A S, and Zimmerman G A. Neutrophils alter the inflammatory milieu by signal-dependent translation of constitutive messenger RNAs. Proceedings of the National Academy of Sciences of the United States of America. 2004;101(18):7076-81.
60. Predina J, Eruslanov E, Judy B, Kapoor V, Cheng G, Wang LC, Sun J, Moon E K, Fridlender Z G, Albelda S, et al. Changes in the local tumor microenvironment in recurrent cancers may explain the failure of vaccines after surgery. Proceedings of the National Academy of Sciences of the United States of America. 2013;110(5):E415-24.
61. Joseph N, Clark R M, Dizon D S, Lee M S, Goodman A, Boruta D, Jr., Schorge J O, Del Carmen M G, and Growdon W B. Delay in chemotherapy administration impacts survival in elderly patients with epithelial ovarian cancer. Gynecol Oncol. 2015;137(3):401-5.
62. Singh S, Guetzko M, and Resnick K. Preoperative predictors of delay in initiation of adjuvant chemotherapy in patients undergoing primary debulking surgery for ovarian cancer. Gynecol Oncol. 2016;143(2):241-5.
63. Pillay J, Kamp V M, van Hoffen E, Visser T, Tak T, Lammers J W, Ulfman L H, Leenen L P, Pickkers P, and Koenderman L. A subset of neutrophils in human systemic inflammation inhibits T cell responses through Mac-1. The Journal of clinical investigation. 2012;122(1):327-36.
64. Janols H, Bergenfelz C, Allaoui R, Larsson A M, Ryden L, Bjornsson S, Janciauskiene S, Wullt M, Bredberg A, and Leandersson K. A high frequency of MDSCs in sepsis patients, with the granulocytic subtype dominating in gram-positive cases. J Leukoc Biol. 2014;96(5):685-93.
65. Gustafson M P, Lin Y, Maas M L, Van Keulen V P, Johnston P B, Peikert T, Gastineau D A, and Dietz A B. A method for identification and analysis of non-overlapping myeloid immunophenotypes in humans. PloS one. 2015;10(3):e0121546.
66. Matsuzaki J, Tsuji T, Luescher I F, Shiku H, Mineno J, Okamoto S, Old L J, Shrikant P, Gnjatic S, and Odunsi K. Direct tumor recognition by a human CD4(+) T-cell subset potently mediates tumor growth inhibition and orchestrates anti-tumor immune responses. Sci Rep. 2015;5(14896.

While the invention has been described through illustrative examples, routine modifications will be apparent to those skilled in the art, which modifications are intended to be within the scope of the invention.

1. A method of providing a treatment for a cancer patient with one or more drugs that inhibit formation of immunosuppressive neutrophils, the method comprising selecting the patient based on a positive result obtained by:
i) exposing a biological sample from the patient to normal neutrophils, and subsequently ii) exposing the neutrophils from i) with T cells, and measuring activation of the T cells, where reduced activation of the T cells relative to a control comprises the positive result and an indication that the individual has the immunosuppressive neutrophils, and administering the drug to the individual.

2. The method of claim 1, wherein the biological sample comprises a pleural effusion or a sample comprising ascites or ascites supernatant.

3. The method of claim 1, wherein the biological sample comprises the ascites supernatant, and wherein the individual has ovarian cancer.

4. The method of claim 1, wherein the drug administered to the individual inhibits SNARE-dependent exocytosis, or inhibits NADPH oxidase, or inhibits complement C3 signaling.

5. The method of claim 4, wherein the drug administered to the individual comprises a SNARE domain of syntaxin-4 or an N-terminal domain of SNAP23.

6. The method of claim 4, wherein the drug that is administered to the individual inhibits C3 signaling and comprises a peptide that selectively binds to native C3, and/or to C3 bioactive fragments selected from C3b, iC3b and C3c.

7. The method of claim 6, wherein the drug comprises compstatin, compstatin derivative Cp40, PEGylated Cp40, or AMY-101.

8. The method of claim 7, further comprising administering to the individual an immune checkpoint inhibitor.

9. The method of claim 8, wherein the drug that inhibits formation of immunosuppressive neutrophils increases the efficacy of the immune checkpoint inhibitor, and/or wherein the combination of the drug that inhibits formation of immunosuppressive neutrophils and the immune checkpoint inhibitor is more effective in treating the cancer than administering either the drug or the immune checkpoint inhibitor alone.

10. A method for determining that a cancer patient has immunosuppressive neutrophils, the method comprising:
i) exposing a biological sample from the patient to normal neutrophils, and subsequently ii) exposing the neutrophils from i) with T cells, and measuring activation of the T cells, where reduced activation of the T cells relative to a control comprises an indication that the individual has the immunosuppressive neutrophils.

11. The method of claim 10, wherein the indication that the individual has the immunosuppressive neutrophils is a candidate to receive a drug that inhibits SNARE-dependent exocytosis, or inhibits NADPH oxidase, or inhibits complement C3 signaling.

12. The method of claim 10, wherein the biological sample comprises a pleural effusion, or ascites or ascites supernatant.

13. The method of claim 12, wherein the biological sample comprises the ascites supernatant, and wherein the individual has ovarian cancer.
